SEQUENCE LISTING (291 sequences)

SEQ ID NO: 1 - DNA, 942 bp, Artificial Sequence
  Synthetic construct
gaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatc

SEQ ID NO: 2 - DNA, 942 bp, Artificial Sequence; identical to SEQ ID NO: 1
  Synthetic construct

SEQ ID NO: 3 - DNA, 942 bp, Artificial Sequence; identical to SEQ ID NO: 1
  Synthetic construct

SEQ ID NO: 4 - DNA, 942 bp, Artificial Sequence
  Synthetic construct
gaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagattgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatc

SEQ ID NO: 5 - DNA, 951 bp, Artificial Sequence
  Synthetic construct
acaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagc

SEQ ID NO: 6 - DNA, 942 bp, Artificial Sequence
  Synthetic construct
gaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccata

SEQ ID NO: 7 - DNA, 942 bp, Artificial Sequence; identical to SEQ ID NO: 6
  Synthetic construct

SEQ ID NO: 8 - DNA, 942 bp, Artificial Sequence; identical to SEQ ID NO: 6
  Synthetic construct

SEQ ID NO: 9 - DNA, 942 bp, Artificial Sequence; identical to SEQ ID NO: 6
  Synthetic construct

SEQ ID NO: 10 - DNA, 942 bp, Artificial Sequence; identical to SEQ ID NO: 6
  Synthetic construct

SEQ ID NO: 11 - DNA, 942 bp, Artificial Sequence; identical to SEQ ID NO: 6
  Synthetic construct

SEQ ID NO: 12 - DNA, 942 bp, Artificial Sequence; identical to SEQ ID NO: 6
  Synthetic construct

SEQ ID NO: 13 - DNA, 942 bp, Artificial Sequence; identical to SEQ ID NO: 6
  Synthetic construct

SEQ ID NO: 14 - DNA, 942 bp, Artificial Sequence; identical to SEQ ID NO: 6
  Synthetic construct

SEQ ID NO: 15 - DNA, 942 bp, Artificial Sequence; identical to SEQ ID NO: 6
  Synthetic construct

SEQ ID NO: 16 - DNA, 942 bp, Artificial Sequence; identical to SEQ ID NO: 6
  Synthetic construct

SEQ ID NO: 17 - DNA, 942 bp, Artificial Sequence; identical to SEQ ID NO: 6
  Synthetic construct

SEQ ID NO: 18 - DNA, 942 bp, Artificial Sequence; identical to SEQ ID NO: 6
  Synthetic construct

SEQ ID NO: 19 - DNA, 942 bp, Artificial Sequence; identical to SEQ ID NO: 6
  Synthetic construct

SEQ ID NO: 20 - DNA, 942 bp, Artificial Sequence; identical to SEQ ID NO: 6
  Synthetic construct

SEQ ID NO: 21 - DNA, 942 bp, Artificial Sequence; identical to SEQ ID NO: 6
  Synthetic construct

SEQ ID NO: 22 - DNA, 942 bp, Artificial Sequence; identical to SEQ ID NO: 6
  Synthetic construct

SEQ ID NO: 23 - DNA, 942 bp, Artificial Sequence; identical to SEQ ID NO: 6
  Synthetic construct

SEQ ID NO: 24 - DNA, 942 bp, Artificial Sequence; identical to SEQ ID NO: 6
  Synthetic construct

SEQ ID NO: 25 - DNA, 918 bp, Artificial Sequence
  Synthetic construct
agtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgaaatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaa

SEQ ID NO: 26 - DNA, 918 bp, Artificial Sequence
  Synthetic construct
agtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaagccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaa

SEQ ID NO: 27 - DNA, 918 bp, Artificial Sequence
  Synthetic construct
agtggttttaggaaaatggcattcccgtcaggcaaagttgaaggatgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaa

SEQ ID NO: 28 - DNA, 918 bp, Artificial Sequence
  Synthetic construct
agtggttttaggaaaatggcattcccgtcaggcaaagttgaaggatgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcggttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataaccttaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaa

SEQ ID NO: 29 - DNA, 918 bp, Artificial Sequence
  Synthetic construct
agcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcag

SEQ ID NO: 30 - DNA, 918 bp, Artificial Sequence
  Synthetic construct
agtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaa

SEQ ID NO: 31 - DNA, 918 bp, Artificial Sequence; identical to SEQ ID NO: 30
  Synthetic construct

SEQ ID NO: 32 - DNA, 918 bp, Artificial Sequence; identical to SEQ ID NO: 30
  Synthetic construct

SEQ ID NO: 33 - DNA, 918 bp, Artificial Sequence; identical to SEQ ID NO: 30
  Synthetic construct

SEQ ID NO: 34 - DNA, 918 bp, Artificial Sequence; identical to SEQ ID NO: 30
  Synthetic construct

SEQ ID NO: 35 - DNA, 918 bp, Artificial Sequence; identical to SEQ ID NO: 30
  Synthetic construct

SEQ ID NO: 36 - DNA, 918 bp, Artificial Sequence; identical to SEQ ID NO: 30
  Synthetic construct

SEQ ID NO: 37 - DNA, 918 bp, Artificial Sequence; identical to SEQ ID NO: 30
  Synthetic construct

SEQ ID NO: 38 - DNA, 918 bp, Artificial Sequence; identical to SEQ ID NO: 30
  Synthetic construct

SEQ ID NO: 39 - DNA, 918 bp, Artificial Sequence; identical to SEQ ID NO: 30
  Synthetic construct

SEQ ID NO: 40 - DNA, 918 bp, Artificial Sequence; identical to SEQ ID NO: 30
  Synthetic construct

SEQ ID NO: 41 - DNA, 918 bp, Artificial Sequence; identical to SEQ ID NO: 30
  Synthetic construct

SEQ ID NO: 42 - DNA, 918 bp, Artificial Sequence; identical to SEQ ID NO: 30
  Synthetic construct

SEQ ID NO: 43 - DNA, 918 bp, Artificial Sequence; identical to SEQ ID NO: 30
  Synthetic construct

SEQ ID NO: 44 - DNA, 918 bp, Artificial Sequence; identical to SEQ ID NO: 30
  Synthetic construct

SEQ ID NO: 45 - DNA, 918 bp, Artificial Sequence; identical to SEQ ID NO: 30
  Synthetic construct

SEQ ID NO: 46 - DNA, 918 bp, Artificial Sequence; identical to SEQ ID NO: 30
  Synthetic construct

SEQ ID NO: 47 - DNA, 918 bp, Artificial Sequence; identical to SEQ ID NO: 30
  Synthetic construct

SEQ ID NO: 48 - DNA, 918 bp, Artificial Sequence; identical to SEQ ID NO: 30
  Synthetic construct

SEQ ID NO: 49 - DNA, 2795 bp, Artificial Sequence
  Synthetic construct
tctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcag

SEQ ID NO: 50 - DNA, 2795 bp, Artificial Sequence
  Synthetic construct
tctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgctgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcag

SEQ ID NO: 51 - DNA, 2795 bp, Artificial Sequence; identical to SEQ ID NO: 49
  Synthetic construct

SEQ ID NO: 52 - DNA, 2795 bp, Artificial Sequence; identical to SEQ ID NO: 49
  Synthetic construct

SEQ ID NO: 53 - DNA, 2798 bp, Artificial Sequence
  Synthetic construct
tctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcag

SEQ ID NO: 54 - DNA, 2795 bp, Artificial Sequence
  Synthetic construct
tcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 55 - DNA, 2795 bp, Artificial Sequence; identical to SEQ ID NO: 54
  Synthetic construct

SEQ ID NO: 56 - DNA, 2795 bp, Artificial Sequence
  Synthetic construct
tcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatctacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 57 - DNA, 2795 bp, Artificial Sequence; identical to SEQ ID NO: 54
  Synthetic construct

SEQ ID NO: 58 - DNA, 2795 bp, Artificial Sequence; identical to SEQ ID NO: 54
  Synthetic construct

SEQ ID NO: 59 - DNA, 2795 bp, Artificial Sequence; identical to SEQ ID NO: 54
  Synthetic construct

SEQ ID NO: 60 - DNA, 2795 bp, Artificial Sequence; identical to SEQ ID NO: 54
  Synthetic construct

SEQ ID NO: 61 - DNA, 2795 bp, Artificial Sequence; identical to SEQ ID NO: 54
  Synthetic construct

SEQ ID NO: 62 - DNA, 2795 bp, Artificial Sequence; identical to SEQ ID NO: 54
  Synthetic construct

SEQ ID NO: 63 - DNA, 2795 bp, Artificial Sequence; identical to SEQ ID NO: 54
  Synthetic construct

SEQ ID NO: 64 - DNA, 2795 bp, Artificial Sequence; identical to SEQ ID NO: 54
  Synthetic construct

SEQ ID NO: 65 - DNA, 2795 bp, Artificial Sequence; identical to SEQ ID NO: 54
  Synthetic construct

SEQ ID NO: 66 - DNA, 2795 bp, Artificial Sequence; identical to SEQ ID NO: 54
  Synthetic construct

SEQ ID NO: 67 - DNA, 2795 bp, Artificial Sequence; identical to SEQ ID NO: 54
  Synthetic construct

SEQ ID NO: 68 - DNA, 2795 bp, Artificial Sequence; identical to SEQ ID NO: 54
  Synthetic construct

SEQ ID NO: 69 - DNA, 2795 bp, Artificial Sequence; identical to SEQ ID NO: 54
  Synthetic construct

SEQ ID NO: 70 - DNA, 2795 bp, Artificial Sequence; identical to SEQ ID NO: 54
  Synthetic construct

SEQ ID NO: 71 - DNA, 2795 bp, Artificial Sequence; identical to SEQ ID NO: 54
  Synthetic construct

SEQ ID NO: 72 - DNA, 2795 bp, Artificial Sequence; identical to SEQ ID NO: 54
  Synthetic construct

SEQ ID NO: 73 - DNA, 1803 bp, Artificial Sequence
  Synthetic construct
gctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaa

SEQ ID NO: 74 - DNA, 1803 bp, Artificial Sequence
  Synthetic construct
gctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgtcacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaa

SEQ ID NO: 75 - DNA, 1803 bp, Artificial Sequence
  Synthetic construct
gctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcatcaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaa

SEQ ID NO: 76 - DNA, 1803 bp, Artificial Sequence; identical to SEQ ID NO: 75
  Synthetic construct

SEQ ID NO: 77 - DNA, 1794 bp, Artificial Sequence
  Synthetic construct
gctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccag

SEQ ID NO: 78 - DNA, 1803 bp, Artificial Sequence
  Synthetic construct
gctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaa

SEQ ID NO: 79 - DNA, 1803 bp, Artificial Sequence; identical to SEQ ID NO: 78
  Synthetic construct

SEQ ID NO: 80 - DNA, 1803 bp, Artificial Sequence; identical to SEQ ID NO: 78
  Synthetic construct

SEQ ID NO: 81 - DNA, 1803 bp, Artificial Sequence; identical to SEQ ID NO: 78
  Synthetic construct

SEQ ID NO: 82 - DNA, 1803 bp, Artificial Sequence; identical to SEQ ID NO: 78
  Synthetic construct

SEQ ID NO: 83 - DNA, 1803 bp, Artificial Sequence; identical to SEQ ID NO: 78
  Synthetic construct

SEQ ID NO: 84 - DNA, 1803 bp, Artificial Sequence; identical to SEQ ID NO: 78
  Synthetic construct

SEQ ID NO: 85 - DNA, 1803 bp, Artificial Sequence; identical to SEQ ID NO: 78
  Synthetic construct

SEQ ID NO: 86 - DNA, 1803 bp, Artificial Sequence; identical to SEQ ID NO: 78
  Synthetic construct

SEQ ID NO: 87 - DNA, 1803 bp, Artificial Sequence; identical to SEQ ID NO: 78
  Synthetic construct

SEQ ID NO: 88 - DNA, 1803 bp, Artificial Sequence; identical to SEQ ID NO: 78
  Synthetic construct

SEQ ID NO: 89 - DNA, 1803 bp, Artificial Sequence; identical to SEQ ID NO: 78
  Synthetic construct

SEQ ID NO: 90 - DNA, 1803 bp, Artificial Sequence; identical to SEQ ID NO: 78
  Synthetic construct

SEQ ID NO: 91 - DNA, 1803 bp, Artificial Sequence; identical to SEQ ID NO: 78
  Synthetic construct

SEQ ID NO: 92 - DNA, 1803 bp, Artificial Sequence; identical to SEQ ID NO: 78
  Synthetic construct

SEQ ID NO: 93 - DNA, 1803 bp, Artificial Sequence; identical to SEQ ID NO: 78
  Synthetic construct

SEQ ID NO: 94 - DNA, 1803 bp, Artificial Sequence
  Synthetic construct
gctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaatactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaa

SEQ ID NO: 95 - DNA, 1803 bp, Artificial Sequence; identical to SEQ ID NO: 78
  Synthetic construct

SEQ ID NO: 96 - DNA, 1803 bp, Artificial Sequence; identical to SEQ ID NO: 78
  Synthetic construct

SEQ ID NO: 97 - DNA, 3768 bp, Artificial Sequence
  Synthetic construct
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttcccttttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcatggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 98 - DNA, 3768 bp, Artificial Sequence
  Synthetic construct
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtctcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 99 - DNA, 3768 bp, Artificial Sequence
  Synthetic construct
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacccatttttaagttgcctcttggtattaacattacaaattttagagccattcttacagcctttttacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaagagatgttgtgagattccctaatattacaaacttgtgtccttttggagaggtttttaatgctactaaattcccttctgtctatgcatgggagaggaaaagaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaagattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgcggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaagaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 100 - DNA, 3768 bp, Artificial Sequence
  Synthetic construct
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacccatttttaagttgcctcttggtattaacattacaaattttagagccattcttacagcctttttacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaagagatgttgtgagattccctaatattacaaacttgtgtccttttggagaggtttttaatgctactaaattcccttctgtctatgcatgggagaggaaaagaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttatgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaagattttggtggttttaatttttcacaaatattacctgaccctctaaagtcaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgcggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 101 - DNA, 4062 bp, Artificial Sequence
  Synthetic construct
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

SEQ ID NO: 102 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaaggtatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 103 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaayaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 104 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 105 - DNA, 3822 bp, Artificial Sequence; identical to SEQ ID NO: 104
  Synthetic construct

SEQ ID NO: 106 - DNA, 3822 bp, Artificial Sequence; identical to SEQ ID NO: 104
  Synthetic construct

SEQ ID NO: 107 - DNA, 3822 bp, Artificial Sequence; identical to SEQ ID NO: 104
  Synthetic construct

SEQ ID NO: 108 - DNA, 3822 bp, Artificial Sequence; identical to SEQ ID NO: 104
  Synthetic construct

SEQ ID NO: 109 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaagttgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 110 - DNA, 3822 bp, Artificial Sequence; identical to SEQ ID NO: 104
  Synthetic construct

SEQ ID NO: 111 - DNA, 3822 bp, Artificial Sequence; identical to SEQ ID NO: 104
  Synthetic construct

SEQ ID NO: 112 - DNA, 3822 bp, Artificial Sequence; identical to SEQ ID NO: 104
  Synthetic construct

SEQ ID NO: 113 - DNA, 3822 bp, Artificial Sequence; identical to SEQ ID NO: 104
  Synthetic construct

SEQ ID NO: 114 - DNA, 3822 bp, Artificial Sequence; identical to SEQ ID NO: 104
  Synthetic construct

SEQ ID NO: 115 - DNA, 3822 bp, Artificial Sequence; identical to SEQ ID NO: 104
  Synthetic construct

SEQ ID NO: 116 - DNA, 3822 bp, Artificial Sequence; identical to SEQ ID NO: 104
  Synthetic construct

SEQ ID NO: 117 - DNA, 3822 bp, Artificial Sequence; identical to SEQ ID NO: 104
  Synthetic construct

SEQ ID NO: 118 - DNA, 3822 bp, Artificial Sequence; identical to SEQ ID NO: 104
  Synthetic construct

SEQ ID NO: 119 - DNA, 3822 bp, Artificial Sequence; identical to SEQ ID NO: 104
  Synthetic construct

SEQ ID NO: 120 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaataaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 121 - DNA, 23 bp, Artificial Sequence
  Synthetic construct
gctgtccgagccggacgagctgc

SEQ ID NO: 122 - DNA, 25 bp, Artificial Sequence
  Synthetic construct
tgctgtccgagccggacgagctgct

SEQ ID NO: 123 - DNA, 27 bp, Artificial Sequence
  Synthetic construct
ctgctgtccgagccggacgagctgctg

SEQ ID NO: 124 - DNA, 29 bp, Artificial Sequence
  Synthetic construct
gctgctgtccgagccggacgagctgctgc

SEQ ID NO: 125 - DNA, 31 bp, Artificial Sequence
  Synthetic construct
tgctgctgtccgagccggacgagctgctgct

SEQ ID NO: 126 - DNA, 33 bp, Artificial Sequence
  Synthetic construct
ctgctgctgtccgagccggacgagctgctgctg

SEQ ID NO: 127 - DNA, 35 bp, Artificial Sequence
  Synthetic construct
gctgctgctgtccgagccggacgagctgctgctgc

SEQ ID NO: 128 - DNA, 37 bp, Artificial Sequence
  Synthetic construct
tgctgctgctgtccgagccggacgagctgctgctgct

SEQ ID NO: 129 - DNA, 39 bp, Artificial Sequence
  Synthetic construct
ctgctgctgctgtccgagccggacgagctgctgctgctg

SEQ ID NO: 130 - DNA, 31 bp, Artificial Sequence; identical to SEQ ID NO: 125
  Synthetic construct
  misc_feature (1)..(1): locked nucleic acid
  misc_feature (2)..(2): locked nucleic acid
  misc_feature (3)..(3): locked nucleic acid
  misc_feature (4)..(4): locked nucleic acid
  misc_feature (5)..(5): locked nucleic acid
  misc_feature (29)..(29): locked nucleic acid
  misc_feature (30)..(30): locked nucleic acid
  misc_feature (31)..(31): locked nucleic acid
  misc_feature (32)..(32): locked nucleic acid
  misc_feature (34)..(34): locked nucleic acid

SEQ ID NO: 131 - DNA, 31 bp, Artificial Sequence; identical to SEQ ID NO: 125
  Synthetic construct
  misc_feature (1)..(1): peptide nucleic acids
  misc_feature (1)..(1): peptide nucleic acids
  misc_feature (2)..(2): peptide nucleic acids
  misc_feature (3)..(3): peptide nucleic acids
  misc_feature (4)..(4): peptide nucleic acids
  misc_feature (5)..(5): peptide nucleic acids
  misc_feature (6)..(6): peptide nucleic acids
  misc_feature (7)..(7): peptide nucleic acids
  misc_feature (8)..(8): peptide nucleic acids
  misc_feature (9)..(9): peptide nucleic acids
  misc_feature (10)..(10): peptide nucleic acids
  misc_feature (12)..(12): peptide nucleic acids
  misc_feature (13)..(13): peptide nucleic acids
  misc_feature (14)..(14): peptide nucleic acids
  misc_feature (15)..(15): peptide nucleic acids
  misc_feature (16)..(16): peptide nucleic acids
  misc_feature (17)..(17): peptide nucleic acids
  misc_feature (18)..(18): peptide nucleic acids
  misc_feature (19)..(19): peptide nucleic acids
  misc_feature (20)..(20): peptide nucleic acids
  misc_feature (21)..(21): peptide nucleic acids
  misc_feature (23)..(23): peptide nucleic acids
  misc_feature (24)..(24): peptide nucleic acids
  misc_feature (25)..(25): peptide nucleic acids
  misc_feature (26)..(26): peptide nucleic acids
  misc_feature (27)..(27): peptide nucleic acids
  misc_feature (28)..(28): peptide nucleic acids
  misc_feature (29)..(29): peptide nucleic acids
  misc_feature (30)..(30): peptide nucleic acids
  misc_feature (31)..(31): peptide nucleic acids
  misc_feature (32)..(32): peptide nucleic acids
  misc_feature (34)..(34): peptide nucleic acids

SEQ ID NO: 132 - DNA, 51 bp, Artificial Sequence
  Synthetic construct
  misc_feature (23)..(23): locked nucleic acids
  misc_feature (24)..(24): locked nucleic acids
  misc_feature (25)..(25): locked nucleic acids
  misc_feature (26)..(26): locked nucleic acids
  misc_feature (27)..(27): locked nucleic acids
  misc_feature (51)..(51): locked nucleic acids
  misc_feature (52)..(52): locked nucleic acids
  misc_feature (53)..(53): locked nucleic acids
  misc_feature (54)..(54): locked nucleic acids
  misc_feature (56)..(56): locked nucleic acids
tttttttttttttttttttttgctgctgtccgagccggacgagctgctgct

SEQ ID NO: 133 - DNA, 83 bp, Artificial Sequence
  Synthetic construct
aaaaaaaaaaaaaaaaaaaaattgctgtattggctctggtgatgcgttaaaggatctcgtatagcagctcagtccactcgaac

SEQ ID NO: 134 - DNA, 63 bp, Artificial Sequence
  Synthetic construct
catagtcaataacgcatcaccagagccaaatgacgacatctgtgcgatgaaacctagcagacc

SEQ ID NO: 135 - DNA, 63 bp, Artificial Sequence
  Synthetic construct
gagatcctatgactatgggtctgctaggtacagtctgtcgcttatgcactagagctgctatac

SEQ ID NO: 136 - DNA, 63 bp, Artificial Sequence
  Synthetic construct
tgtcgtcaaacagcaatgttcgagtggacaagtgcataagcgacagactgatcatcgcacaga

SEQ ID NO: 137 - DNA, 20 bp, Artificial Sequence
  Synthetic construct
gacagtcagccgcatcttct

SEQ ID NO: 138 - DNA, 20 bp, Artificial Sequence
  Synthetic construct
gcgcccaatacgaccaaatc

SEQ ID NO: 139 - DNA, 20 bp, Artificial Sequence
  Synthetic construct
caagatccgccacaacatcg

SEQ ID NO: 140 - DNA, 20 bp, Artificial Sequence
  Synthetic construct
gactgggtgctcaggtagtg

SEQ ID NO: 141 - DNA, 19 bp, Artificial Sequence
  Synthetic construct
tcatgtggtagtgttggtt

SEQ ID NO: 142 - DNA, 19 bp, Artificial Sequence
  Synthetic construct
gatgttgttagacaatgct

SEQ ID NO: 143 - DNA, 19 bp, Artificial Sequence
  Synthetic construct
tgcggtgtaagtgcagccc

SEQ ID NO: 144 - DNA, 19 bp, Artificial Sequence
  Synthetic construct
gtgcggcacaggcactagt

SEQ ID NO: 145 - DNA, 19 bp, Artificial Sequence
  Synthetic construct
acaggcactagtactgatg

SEQ ID NO: 146 - DNA, 19 bp, Artificial Sequence
  Synthetic construct
tacataatcaggatgtaaa

SEQ ID NO: 147 - DNA, 19 bp, Artificial Sequence
  Synthetic construct
gtttctttaaggaaggaag

SEQ ID NO: 148 - DNA, 19 bp, Artificial Sequence
  Synthetic construct
ctttaaggaaggaagttct

SEQ ID NO: 149 - DNA, 19 bp, Artificial Sequence
  Synthetic construct
aaggaaggaagttctgttg

SEQ ID NO: 150 - DNA, 19 bp, Artificial Sequence
  Synthetic construct
aatggggtaaggctagact

SEQ ID NO: 151 - DNA, 19 bp, Artificial Sequence
  Synthetic construct
ggtaaggctagactttatt

SEQ ID NO: 152 - DNA, 19 bp, Artificial Sequence
  Synthetic construct
tgagttatgaggatcaaga

SEQ ID NO: 153 - DNA, 19 bp, Artificial Sequence
  Synthetic construct
tgaggatcaagatgcactt

SEQ ID NO: 154 - DNA, 19 bp, Artificial Sequence
  Synthetic construct
tgaatcttaagtatgccat

SEQ ID NO: 155 - DNA, 19 bp, Artificial Sequence
  Synthetic construct
tatgccattagtgcaaaga

SEQ ID NO: 156 - DNA, 19 bp, Artificial Sequence
  Synthetic construct
gcaaagaatagagctcgca

SEQ ID NO: 157 - DNA, 19 bp, Artificial Sequence
  Synthetic construct
aaagaatagagctcgcacc

SEQ ID NO: 158 - DNA, 19 bp, Artificial Sequence
  Synthetic construct
tcgcaccgtagctggtgtc

SEQ ID NO: 159 - DNA, 19 bp, Artificial Sequence
  Synthetic construct
tctatctgtagtactatga

SEQ ID NO: 160 - DNA, 19 bp, Artificial Sequence
  Synthetic construct
gccgccactagaggagcta

SEQ ID NO: 161 - DNA, 19 bp, Artificial Sequence
  Synthetic construct
cgccactagaggagctact

SEQ ID NO: 162 - DNA, 19 bp, Artificial Sequence
  Synthetic construct
gttggactgagactgacct

SEQ ID NO: 163 - DNA, 19 bp, Artificial Sequence
  Synthetic construct
atcctaatcaggagtatgc

SEQ ID NO: 164 - DNA, 19 bp, Artificial Sequence
  Synthetic construct
ctaatcaggagtatgctga

SEQ ID NO: 165 - DNA, 19 bp, Artificial Sequence
  Synthetic construct
gggaacctgagttttatga

SEQ ID NO: 166 - DNA, 19 bp, Artificial Sequence
  Synthetic construct
agttttatgaggctatgta

SEQ ID NO: 167 - DNA, 19 bp, Artificial Sequence
  Synthetic construct
ctttgaaaaaggtgactat

SEQ ID NO: 168 - DNA, 19 bp, Artificial Sequence
  Synthetic construct
tctcagatgagttttctag

SEQ ID NO: 169 - DNA, 19 bp, Artificial Sequence
  Synthetic construct
gagttttctagcaatgttg

SEQ ID NO: 170 - DNA, 19 bp, Artificial Sequence
  Synthetic construct
gtactggtaagagtcattt

SEQ ID NO: 171 - DNA, 19 bp, Artificial Sequence
  Synthetic construct
ttctctatgagaaccaaaa

SEQ ID NO: 172 - DNA, 31 bp, Artificial Sequence
  Synthetic construct
aaccaacatccgagccggacgaaccacatga

SEQ ID NO: 173 - DNA, 31 bp, Artificial Sequence
  Synthetic construct
agcattgttccgagccggacgaaacaacatc

SEQ ID NO: 174 - DNA, 31 bp, Artificial Sequence
  Synthetic construct
gggctgcatccgagccggacgatacaccgca

SEQ ID NO: 175 - DNA, 31 bp, Artificial Sequence
  Synthetic construct
actagtgctccgagccggacgagtgccgcac

SEQ ID NO: 176 - DNA, 31 bp, Artificial Sequence
  Synthetic construct
catcagtatccgagccggacgaagtgcctgt

SEQ ID NO: 177 - DNA, 31 bp, Artificial Sequence
  Synthetic construct
tttacatctccgagccggacgagattatgta

SEQ ID NO: 178 - DNA, 31 bp, Artificial Sequence
  Synthetic construct
cttccttctccgagccggacgataaagaaac

SEQ ID NO: 179 - DNA, 31 bp, Artificial Sequence
  Synthetic construct
agaacttctccgagccggacgatccttaaag

SEQ ID NO: 180 - DNA, 31 bp, Artificial Sequence
  Synthetic construct
caacagaatccgagccggacgatccttcctt

SEQ ID NO: 181 - DNA, 31 bp, Artificial Sequence
  Synthetic construct
agtctagctccgagccggacgataccccatt

SEQ ID NO: 182 - DNA, 31 bp, Artificial Sequence
  Synthetic construct
aataaagttccgagccggacgaagccttacc

SEQ ID NO: 183 - DNA, 31 bp, Artificial Sequence
  Synthetic construct
tcttgatctccgagccggacgacataactca

SEQ ID NO: 184 - DNA, 31 bp, Artificial Sequence
  Synthetic construct
aagtgcattccgagccggacgatgatcctca

SEQ ID NO: 185 - DNA, 31 bp, Artificial Sequence
  Synthetic construct
atggcatatccgagccggacgataagattca

SEQ ID NO: 186 - DNA, 31 bp, Artificial Sequence
  Synthetic construct
tctttgcatccgagccggacgaaatggcata

SEQ ID NO: 187 - DNA, 31 bp, Artificial Sequence
  Synthetic construct
tgcgagcttccgagccggacgaattctttgc

SEQ ID NO: 188 - DNA, 31 bp, Artificial Sequence
  Synthetic construct
ggtgcgagtccgagccggacgactattcttt

SEQ ID NO: 189 - DNA, 31 bp, Artificial Sequence
  Synthetic construct
gacaccagtccgagccggacgaacggtgcga

SEQ ID NO: 190 - DNA, 31 bp, Artificial Sequence
  Synthetic construct
tcatagtatccgagccggacgaacagataga

SEQ ID NO: 191 - DNA, 31 bp, Artificial Sequence
  Synthetic construct
tagctccttccgagccggacgaagtggcggc

SEQ ID NO: 192 - DNA, 31 bp, Artificial Sequence
  Synthetic construct
agtagctctccgagccggacgactagtggcg

SEQ ID NO: 193 - DNA, 31 bp, Artificial Sequence
  Synthetic construct
aggtcagttccgagccggacgacagtccaac

SEQ ID NO: 194 - DNA, 31 bp, Artificial Sequence
  Synthetic construct
gcatactctccgagccggacgagattaggat

SEQ ID NO: 195 - DNA, 31 bp, Artificial Sequence
  Synthetic construct
tcagcatatccgagccggacgacctgattag

SEQ ID NO: 196 - DNA, 31 bp, Artificial Sequence
  Synthetic construct
tcataaaatccgagccggacgacaggttccc

SEQ ID NO: 197 - DNA, 31 bp, Artificial Sequence
  Synthetic construct
tacatagctccgagccggacgacataaaact

SEQ ID NO: 198 - DNA, 31 bp, Artificial Sequence
  Synthetic construct
atagtcactccgagccggacgattttcaaag

SEQ ID NO: 199 - DNA, 31 bp, Artificial Sequence
  Synthetic construct
ctagaaaatccgagccggacgacatctgaga

SEQ ID NO: 200 - DNA, 31 bp, Artificial Sequence
  Synthetic construct
caacattgtccgagccggacgaagaaaactc

SEQ ID NO: 201 - DNA, 31 bp, Artificial Sequence
  Synthetic construct
aaatgacttccgagccggacgataccagtac

SEQ ID NO: 202 - DNA, 31 bp, Artificial Sequence
  Synthetic construct
ttttggtttccgagccggacgacatagagaa

SEQ ID NO: 203 - DNA, 8 bp, Artificial Sequence
  Synthetic construct
aaccaaca

SEQ ID NO: 204 - DNA, 8 bp, Artificial Sequence
  Synthetic construct
agcattgt

SEQ ID NO: 205 - DNA, 8 bp, Artificial Sequence
  Synthetic construct
gggctgca

SEQ ID NO: 206 - DNA, 8 bp, Artificial Sequence
  Synthetic construct
actagtgc

SEQ ID NO: 207 - DNA, 8 bp, Artificial Sequence
  Synthetic construct
catcagta

SEQ ID NO: 208 - DNA, 8 bp, Artificial Sequence
  Synthetic construct
tttacatc

SEQ ID NO: 209 - DNA, 8 bp, Artificial Sequence
  Synthetic construct
cttccttc

SEQ ID NO: 210 - DNA, 8 bp, Artificial Sequence
  Synthetic constuct
agaacttc

SEQ ID NO: 211 - DNA, 8 bp, Artificial Sequence
  Synthetic constuct
caacagaa

SEQ ID NO: 212 - DNA, 8 bp, Artificial Sequence
  Synthetic constuct
agtctagc

SEQ ID NO: 213 - DNA, 8 bp, Artificial Sequence
  Synthetic constuct
aataaagt

SEQ ID NO: 214 - DNA, 8 bp, Artificial Sequence
  Synthetic constuct
tcttgatc

SEQ ID NO: 215 - DNA, 8 bp, Artificial Sequence
  Synthetic constuct
aagtgcat

SEQ ID NO: 216 - DNA, 8 bp, Artificial Sequence
  Synthetic constuct
atggcata

SEQ ID NO: 217 - DNA, 8 bp, Artificial Sequence
  Synthetic constuct
tctttgca

SEQ ID NO: 218 - DNA, 8 bp, Artificial Sequence
  Synthetic constuct
tgcgagct

SEQ ID NO: 219 - DNA, 8 bp, Artificial Sequence
  Synthetic construct
ggtgcgag

SEQ ID NO: 220 - DNA, 8 bp, Artificial Sequence
  Synthetic constuct
gacaccag

SEQ ID NO: 221 - DNA, 8 bp, Artificial Sequence
  Synthetic construct
tcatagta

SEQ ID NO: 222 - DNA, 8 bp, Artificial Sequence
  Synthetic construct
tagctcct

SEQ ID NO: 223 - DNA, 8 bp, Artificial Sequence
  Synthetic construct
agtagctc

SEQ ID NO: 224 - DNA, 8 bp, Artificial Sequence
  Synthetic construct
aggtcagt

SEQ ID NO: 225 - DNA, 8 bp, Artificial Sequence
  Synthetic construct
gcatactc

SEQ ID NO: 226 - DNA, 8 bp, Artificial Sequence
  Synthetic construct
tcagcata

SEQ ID NO: 227 - DNA, 8 bp, Artificial Sequence
  Synthetic construct
tcataaaa

SEQ ID NO: 228 - DNA, 8 bp, Artificial Sequence
  Synthetic construct
tacatagc

SEQ ID NO: 229 - DNA, 8 bp, Artificial Sequence
  Synthetic construct
atagtcac

SEQ ID NO: 230 - DNA, 8 bp, Artificial Sequence
  Synthetic construct
ctagaaaa

SEQ ID NO: 231 - DNA, 8 bp, Artificial Sequence
  Synthetic construct
caacattg

SEQ ID NO: 232 - DNA, 8 bp, Artificial Sequence
  Synthetic construct
aaatgact

SEQ ID NO: 233 - DNA, 8 bp, Artificial Sequence
  Synthetic construct
ttttggtt

SEQ ID NO: 234 - DNA, 9 bp, Artificial Sequence
  Synthetic construct
accacatga

SEQ ID NO: 235 - DNA, 9 bp, Artificial Sequence
  Synthetic construct
aacaacatc

SEQ ID NO: 236 - DNA, 9 bp, Artificial Sequence
  Synthetic construct
tacaccgca

SEQ ID NO: 237 - DNA, 9 bp, Artificial Sequence
  Synthetic construct
gtgccgcac

SEQ ID NO: 238 - DNA, 9 bp, Artificial Sequence
  Synthetic construct
agtgcctgt

SEQ ID NO: 239 - DNA, 9 bp, Artificial Sequence
  Synthetic construct
gattatgta

SEQ ID NO: 240 - DNA, 9 bp, Artificial Sequence
  Synthetic construct
taaagaaac

SEQ ID NO: 241 - DNA, 9 bp, Artificial Sequence
  Synthetic construct
tccttaaag

SEQ ID NO: 242 - DNA, 9 bp, Artificial Sequence
  Synthetic construct
tccttcctt

SEQ ID NO: 243 - DNA, 9 bp, Artificial Sequence
  Synthetic construct
taccccatt

SEQ ID NO: 244 - DNA, 9 bp, Artificial Sequence
  Synthetic construct
agccttacc

SEQ ID NO: 245 - DNA, 9 bp, Artificial Sequence
  Synthetic construct
cataactca

SEQ ID NO: 246 - DNA, 9 bp, Artificial Sequence
  Synthetic construct
tgatcctca

SEQ ID NO: 247 - DNA, 9 bp, Artificial Sequence
  Synthetic construct
taagattca

SEQ ID NO: 248 - DNA, 9 bp, Artificial Sequence
  Synthetic construct
aatggcata

SEQ ID NO: 249 - DNA, 9 bp, Artificial Sequence
  Synthetic construct
attctttgc

SEQ ID NO: 250 - DNA, 9 bp, Artificial Sequence
  Synthetic construct
ctattcttt

SEQ ID NO: 251 - DNA, 9 bp, Artificial Sequence
  Synthetic construct
acggtgcga

SEQ ID NO: 252 - DNA, 9 bp, Artificial Sequence
  Synthetic construct
acagataga

SEQ ID NO: 253 - DNA, 9 bp, Artificial Sequence
  Synthetic construct
agtggcggc

SEQ ID NO: 254 - DNA, 9 bp, Artificial Sequence
  Synthetic construct
ctagtggcg

SEQ ID NO: 255 - DNA, 9 bp, Artificial Sequence
  Synthetic construct
cagtccaac

SEQ ID NO: 256 - DNA, 9 bp, Artificial Sequence
  Synthetic construct
gattaggat

SEQ ID NO: 257 - DNA, 9 bp, Artificial Sequence
  Synthetic construct
cctgattag

SEQ ID NO: 258 - DNA, 9 bp, Artificial Sequence
  Synthetic construct
caggttccc

SEQ ID NO: 259 - DNA, 9 bp, Artificial Sequence
  Synthetic construct
cataaaact

SEQ ID NO: 260 - DNA, 9 bp, Artificial Sequence
  Synthetic construct
ttttcaaag

SEQ ID NO: 261 - DNA, 9 bp, Artificial Sequence
  Synthetic construct
catctgaga

SEQ ID NO: 262 - DNA, 9 bp, Artificial Sequence
  Synthetic construct
agaaaactc

SEQ ID NO: 263 - DNA, 9 bp, Artificial Sequence
  Synthetic construct
taccagtac

SEQ ID NO: 264 - DNA, 9 bp, Artificial Sequence
  Synthetic construct
catagagaa

SEQ ID NO: 265 - DNA, 4 bp, Artificial Sequence
  Synthetic construct
gctg

SEQ ID NO: 266 - DNA, 5 bp, Artificial Sequence
  Synthetic construct
gctgc

SEQ ID NO: 267 - DNA, 5 bp, Artificial Sequence
  Synthetic construct
tgctg

SEQ ID NO: 268 - DNA, 6 bp, Artificial Sequence
  Synthetic construct
gctgct

SEQ ID NO: 269 - DNA, 6 bp, Artificial Sequence
  Synthetic construct
ctgctg

SEQ ID NO: 270 - DNA, 7 bp, Artificial Sequence
  Synthetic construct
gctgctg

SEQ ID NO: 271 - DNA, 7 bp, Artificial Sequence
  Synthetic construct
gctgctg

SEQ ID NO: 272 - DNA, 8 bp, Artificial Sequence
  Synthetic construct
gctgctgc

SEQ ID NO: 273 - DNA, 8 bp, Artificial Sequence
  Synthetic construct
tgctgctg

SEQ ID NO: 274 - DNA, 9 bp, Artificial Sequence
  Synthetic construct
gctgctgct

SEQ ID NO: 275 - DNA, 9 bp, Artificial Sequence
  Synthetic construct
ctgctgctg

SEQ ID NO: 276 - DNA, 10 bp, Artificial Sequence
  Synthetic construct
gctgctgctg

SEQ ID NO: 277 - DNA, 10 bp, Artificial Sequence
  Synthetic construct
gctgctgctg

SEQ ID NO: 278 - DNA, 11 bp, Artificial Sequence
  Synthetic construct
gctgctgctgc

SEQ ID NO: 279 - DNA, 11 bp, Artificial Sequence
  Synthetic construct
tgctgctgctg

SEQ ID NO: 280 - DNA, 12 bp, Artificial Sequence
  Synthetic construct
gctgctgctgct

SEQ ID NO: 281 - DNA, 12 bp, Artificial Sequence
  Synthetic construct
ctgctgctgctg

SEQ ID NO: 282 - DNA, 13 bp, Artificial Sequence
  Synthetic construct
gctgctgctgctg

SEQ ID NO: 283 - DNA, 8 bp, Artificial Sequence
  Synthetic construct
  misc_feature (1)..(1): locked nucleic acids
  misc_feature (2)..(2): locked nucleic acids
  misc_feature (3)..(3): locked nucleic acids
  misc_feature (4)..(4): locked nucleic acids
  misc_feature (5)..(5): locked nucleic acids
tgctgctg

SEQ ID NO: 284 - DNA, 9 bp, Artificial Sequence
  Synthetic construct
  misc_feature (5)..(5): locked nucleic acids
  misc_feature (6)..(6): locked nucleic acids
  misc_feature (7)..(7): locked nucleic acids
  misc_feature (8)..(8): locked nucleic acids
  misc_feature (9)..(9): locked nucleic acids
gctgctgct

SEQ ID NO: 285 - DNA, 8 bp, Artificial Sequence
  Synthetic construct
  misc_feature (1)..(1): peptide nucleic acids
  misc_feature (2)..(2): peptide nucleic acids
  misc_feature (3)..(3): peptide nucleic acids
  misc_feature (4)..(4): peptide nucleic acids
  misc_feature (5)..(5): peptide nucleic acids
  misc_feature (6)..(6): peptide nucleic acids
  misc_feature (7)..(7): peptide nucleic acids
  misc_feature (8)..(8): peptide nucleic acids
tgctgctg

SEQ ID NO: 286 - DNA, 9 bp, Artificial Sequence
  Synthetic construct
  misc_feature (1)..(1): peptide nucleic acids
  misc_feature (2)..(2): peptide nucleic acids
  misc_feature (3)..(3): peptide nucleic acids
  misc_feature (4)..(4): peptide nucleic acids
  misc_feature (5)..(5): peptide nucleic acids
  misc_feature (6)..(6): peptide nucleic acids
  misc_feature (7)..(7): peptide nucleic acids
  misc_feature (8)..(8): peptide nucleic acids
  misc_feature (9)..(9): peptide nucleic acids
gctgctgct

SEQ ID NO: 287 - DNA, 14 bp, Artificial Sequence
  Synthetic construct
tccgagccggacga

SEQ ID NO: 288 - DNA, 27 bp, Artificial Sequence
  Synthetic construct
cagcagcagcagcagcagcagcagcag

SEQ ID NO: 289 - DNA, 19 bp, Artificial Sequence
  Synthetic construct
agcagcagcagcagcagca

SEQ ID NO: 290 - DNA, 150 bp, Artificial Sequence
  Synthetic construct
agtttttctcatggtgtatttattcttttaagttttgttttttaaatatacttcacttttgaatgtttcagacagcagcaaaagcagcaacagcagcagcagcagcagcagcagggggacatatcaggacagagttcacatccatgtgaa

SEQ ID NO: 291 - DNA, 15 bp, Artificial Sequence
  Synthetic construct
ggctagctacaacga

(19) United States
(12) Patent Application Publication
(10) Pub. No.: US 2023/0174982 A1
(43) Pub. Date: Jun. 8, 2023

(54) ENGINEERED NUCLEIC ACIDS AND USES THEREOF

(71) Applicant: THE METHODIST HOSPITAL SYSTEM, Houston, TX (US)

(72) Inventors: Nan ZHANG, Houston, TX (US); Tetsuo ASHIZAWA, Seabrook, TX (US)

(21) Appl. No.: 17/910,622

(22) PCT Filed: Mar. 9, 2021
(86) PCT No.: PCT/US2021/021491

§ 371 Date: Sep. 9, 2022

Related U.S. Application Data
(60) Provisional application No. 62/987,072, filed on Mar. 9, 2020.
(60) Provisional application No. 62/990,561, filed on Mar. 17, 2020.

(51) Int. Cl. C12N 15/113; A61P 31/14

(52) U.S. Cl. CPC C12N 15/113; A61P 31/14; C12N 2310/127; C12N 2310/3181; C12N 2310/3231; C12N 2320/30

(57) ABSTRACT

The present disclosure relates to engineered nucleic acids that target CAG repeat sequences or viral polynucleotides. The present disclosure also relates to the uses of the engineered nucleic acids for treating polyglutamine diseases or a viral infection.

### CROSS-REFERENCE TO RELATED APPLICATIONS

This application claims the benefit of U.S. Provisional Application No. 62/987,072, filed Mar. 9, 2020, and U.S. Provisional Application No. 62/990,561, filed Mar. 17, 2020, which are expressly incorporated herein by reference in their entireties.

### STATEMENT REGARDING FEDERALLY SPONSORED RESEARCH

This invention was made with government support under Grant No. R01 NS083564 and R01 NS083564 awarded by the National Institutes of Health. The government has certain rights in the invention.

### FIELD

The present disclosure relates to engineered nucleic acids and uses thereof.

### REFERENCE TO SEQUENCE LISTING

The Sequence Listing submitted Mar. 9, 2021 as a text file named “10063-051WO1_2021_03_09_Sequence_Listing.txt,” created on Mar. 9, 2021, and having a size of 374,000 bytes is hereby incorporated by reference pursuant to 37 C.F.R. § 1.52(e)(5).

### BACKGROUND

Polyglutamine diseases are a group of neurodegenerative disorders caused by the expansion of CAG repeats that encode glutamine. The leading therapeutic strategies for targeting CAG repeats reply on CRISPR-Cas, siRNA or ASOs. Despite the fact that CRISPR-Cas has revolutionized gene editing, it cannot be used without careful considerations. With the off-target effects still unclear, DNA editing or damage induced by CRISPR-Cas is permanent, especially large unwanted genomic deletions and complex rearrangements have been recently observed. Secondly, gene editing is prone to mosaicism in adults, that is, not all cells are edited at the same time or to the same extents. Given that polyglutamine diseases manifest mostly in late adulthood, obtaining a relatively homogenous editing in embryos is unattainable. Thirdly, the CRISPR editing efficiency may be hindered by the tumor suppressor p53 to prevent double-strand DNA breaks. Cells that favor editing may possess a dysfunctional p53 and later give rise to cancer. In addition, the editing efficiency is affected by the orientation of the CRISPR-Cas complex on the target gene and with respect to the RNA polymerase. No approach using RNA-base agents has reached human trials. For instance, siRNA does not distribute well into brain tissue and shows limited efficiency that requires the RISC complex assembly, possibly due to its intrinsic instability. Given that the RNA-induced silencing complex (RISC) complexes are mainly located in the cytosol, RNA in the nucleus is largely inaccessible. ASOs are the best current option of treating polyglutamine diseases. They are short DNA fragments that bind to target RNA through base pairing. However, this approach relies on the availability of RNase H in the cell. What is needed are compositions and methods for treating polyglutamine diseases. The compositions and methods disclosed herein address these and other needs

### SUMMARY

In some aspects, disclosed herein is an engineered nucleic acid, comprising:
a first binding arm,
a catalytic core domain, and
a second binding arm,
wherein the catalytic core domain is in between the first binding arm and the second binding arm, and wherein the first binding arm and the second binding arm are complementary to a CAG repeat sequence.

In some embodiments, the catalytic core domain comprises a sequence at least 80% identical to SEQ ID NO: 287.

In some embodiments, the catalytic core domain comprises SEQ ID NO: 287. In some embodiments, the first binding arm and the second binding arm comprise at least 3 nucleotides. In some embodiments, the first and the second binding arms comprise 6, 7, 8, 9, 10, 11, 12, or 13 nucleotides. In some embodiments, the first binding arm and the second binding arm comprise 9 nucleotides.

In some embodiments, the first binding arm and the second binding arm are complementary to CAG repeat sequence. In some embodiments, the first binding arm and the second binding arm comprise a sequence at least 80% identical to SEQ ID NO: 265-286.

In some embodiments, at least one nucleotide of the engineered nucleic acid is a chemically modified ribose. In some embodiments, at least one nucleotide at the 5′-terminus and at least one nucleotide at the 3′-termius of the engineered nucleic acid are chemically modified. In some embodiments, the chemically modified ribose is a locked nucleic acid (LNA) or a peptide nucleic acid (PNA).

In some embodiments, the nucleic acid comprises a sequence at least 80% identical to SEQ ID NOs: 121-132.

In some aspects, disclosed herein are methods of treating a polyglutamine disease in subject in need thereof comprising administering to the subject in need thereof a therapeutically effective amount of the engineered nucleic acid using the engineered nucleic acid of any preceding aspect.

In some embodiments, the polyglutamine disease-related polynucleotide comprises an increased level of CAG repeat sequence as compared to a reference control. In some embodiments, the polyglutamine disease-related polynucleotide is selected from a huntingtin (HTT) polynucleotide, an ATXN1 polynucleotide, an ATXN2 polynucleotide, an ATXN7 polynucleotide, a TATA binding protein-coding polynucleotide, an androgen receptor (AR) polynucleotide, or an atrophin 1 (ATN1) polynucleotide.

In some embodiments, the polyglutamine disease is selected from Huntington’s disease (HD), spinocerebellar ataxias (SCA) type 1, SCA type 2, SCA type 3, SCA type 6, SCA type 7, SCA type 17, Dentatorubral-pallidoluysian atrophy (DRPLA), and spinobulbar muscular atrophy (SBMA).

In some aspects, disclosed herein is an engineered nucleic acid, comprising:
a first binding arm,
a catalytic core domain, and
a second binding arm,
wherein the catalytic core domain is in between the first binding arm and the second binding arm, and wherein the first binding arm and the second binding arm are complementary to a SARS-CoV-2 polynucleotide.

In some embodiments, the catalytic core domain comprises a sequence at least 80% identical to SEQ ID NO: 287.

In some embodiments, the catalytic core domain comprises SEQ ID NO: 287. In some embodiments, the first binding arm and the second binding arm comprise at least 3 nucleotides. In some embodiments, the first and the second binding arms comprise 6, 7, 8, 9, 10, 11, 12, or 13 nucleotides. In some embodiments, the first binding arm and the second binding arm comprise 9 nucleotides.

In some embodiments, the first binding arm and the second binding arm are complementary to a SARS-CoV-2 polynucleotide. In some embodiments, the first binding arm and the second binding arm comprise a sequence at least 80% identical to SEQ ID NO: 203-264.

In some embodiments, at least one nucleotide of the engineered nucleic acid is a chemically modified ribose. In some embodiments, at least one nucleotide at the 5′-terminus and at least one nucleotide at the 3′-termius of the engineered nucleic acid are chemically modified. In some embodiments, the chemically modified ribose is a locked nucleic acid (LNA) or a peptide nucleic acid (PNA).

In some embodiments, the nucleic acid comprises a sequence at least 80% identical to SEQ ID NOs: 172-202.

In some aspects, disclosed herein are methods of treating a SARS-CoV-2 infection in a subject in need thereof, comprising administering to the subject in need thereof a therapeutically effective amount of the engineered nucleic acid of any preceding aspect. In some embodiments, the engineered nucleic acid decreases a level of a SARS-CoV-2 polynucleotide.

### BRIEF DESCRIPTION OF THE DRAWINGS

The accompanying figures, which are incorporated in and constitute a part of this specification, illustrate several aspects described below.

FIGS. 1A-1B show DNAzyme design and biochemical activity. FIG. 1A shows designs of 8-17 Dzs with different RNA binding arms’ lengths. Locked nucleic acids (LNAs) are underlined. The Peptide nucleic acid (PNA) is

*T*G*C*T*G*C*T*G*T*C*C*G*A*G*C*C*G*G*A*C*G*A*G*C*T
*G*C*T*G*C*T (SEQ ID NO: 131).

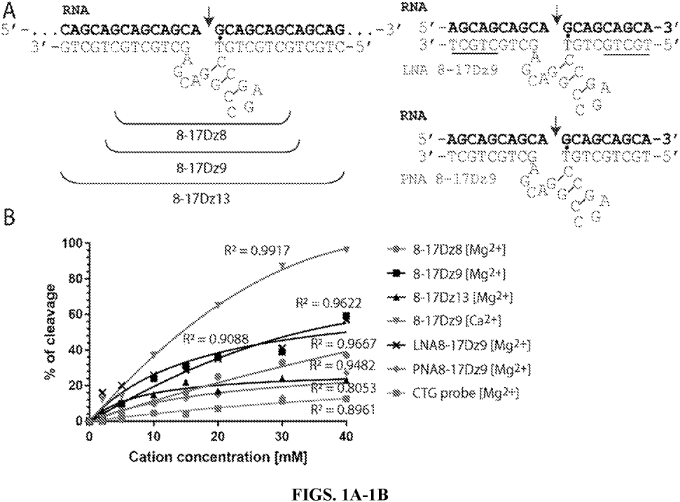

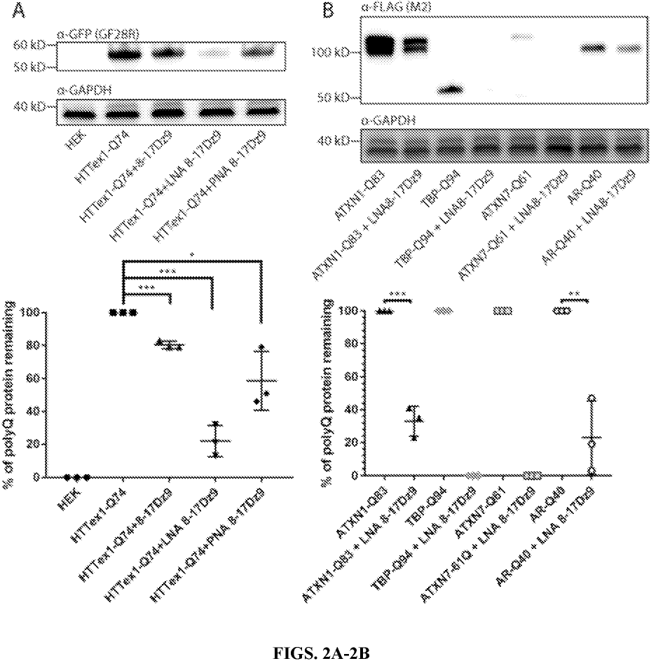

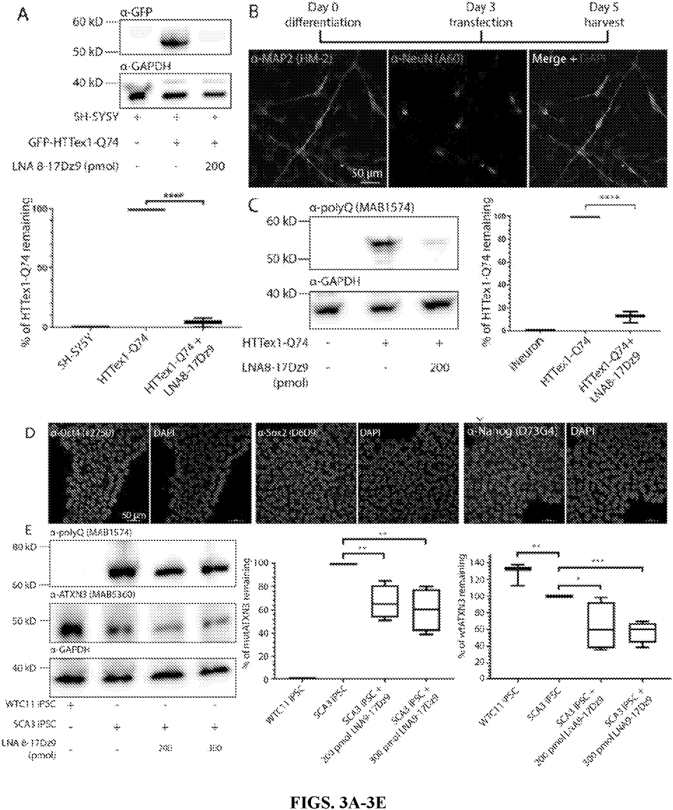

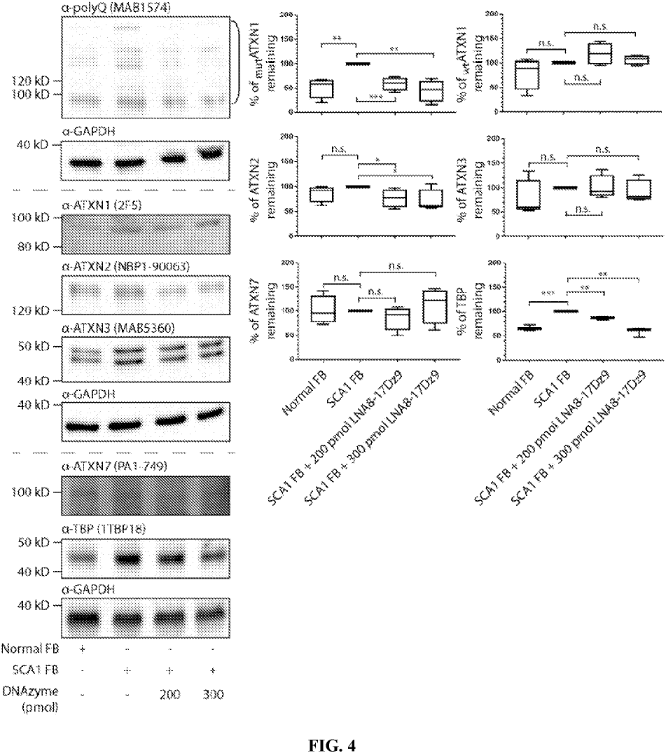

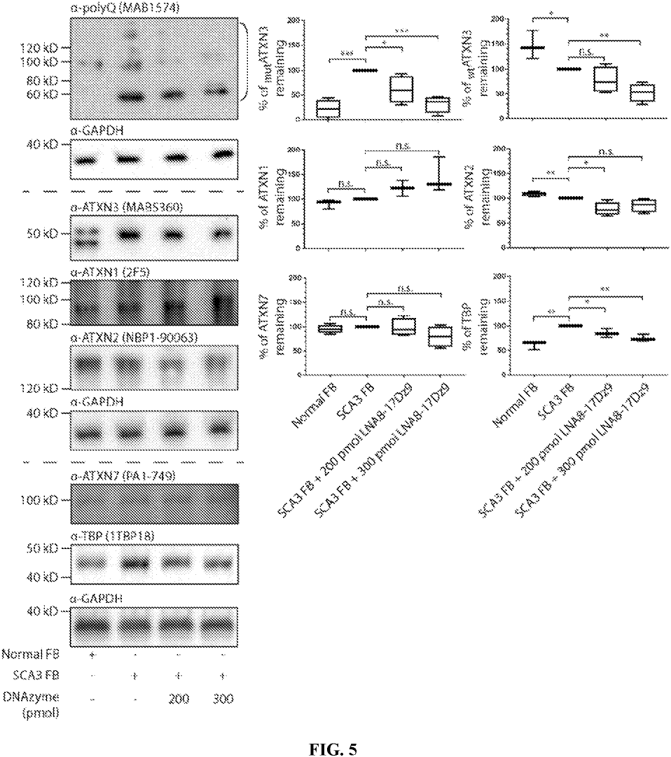

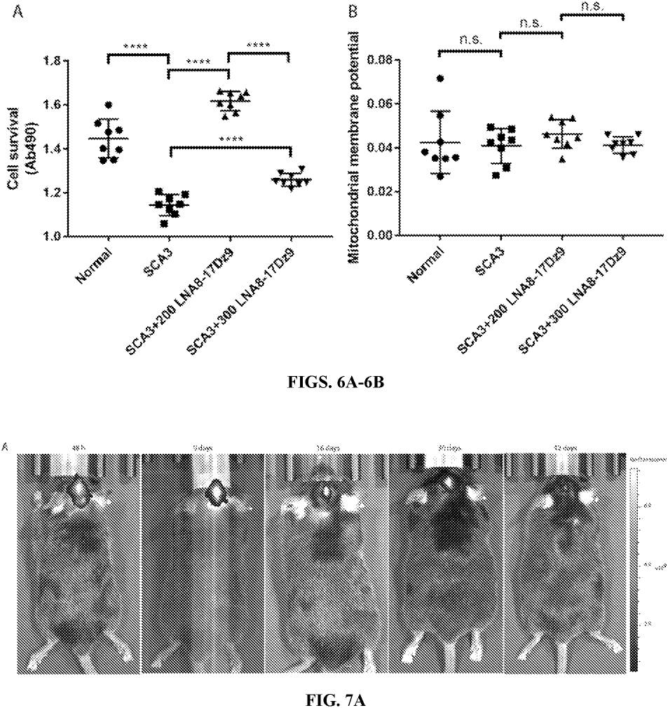

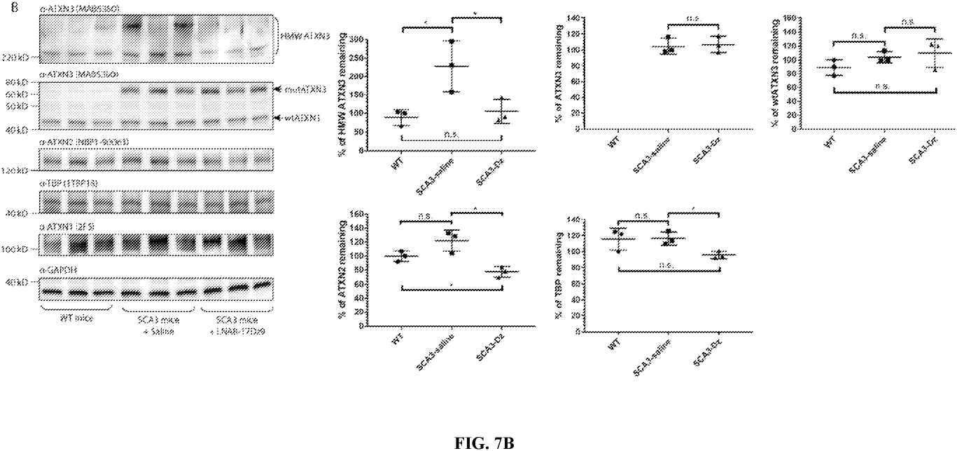

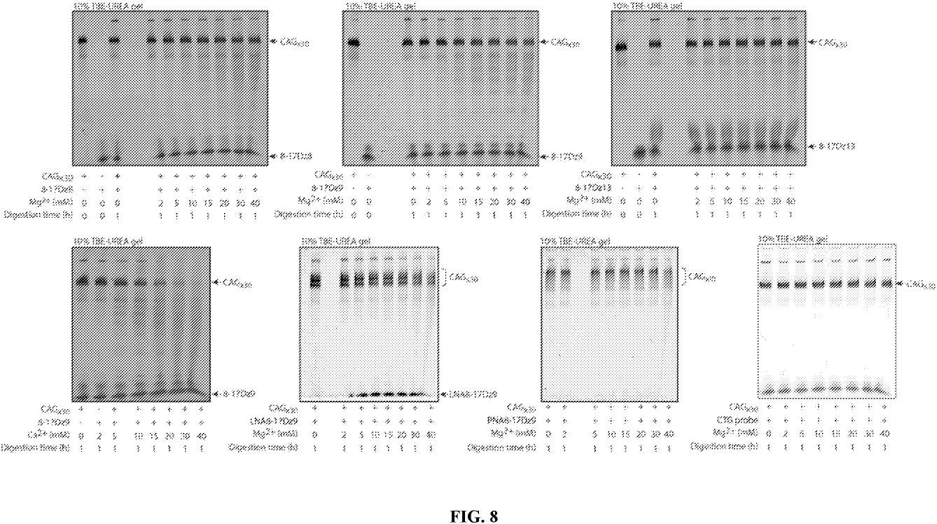

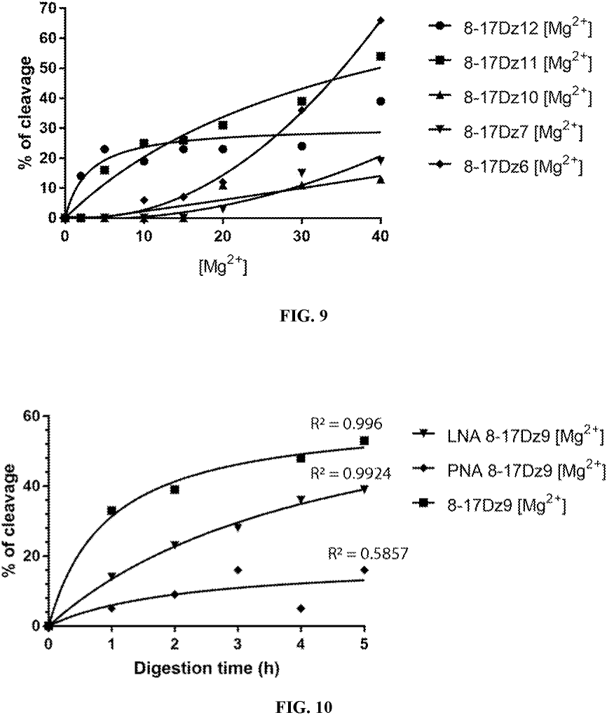

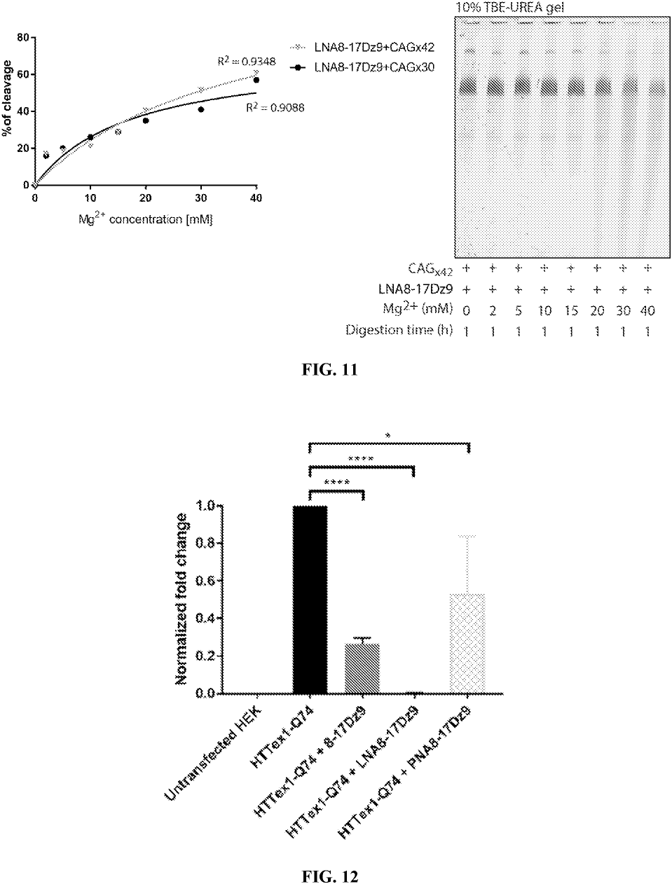

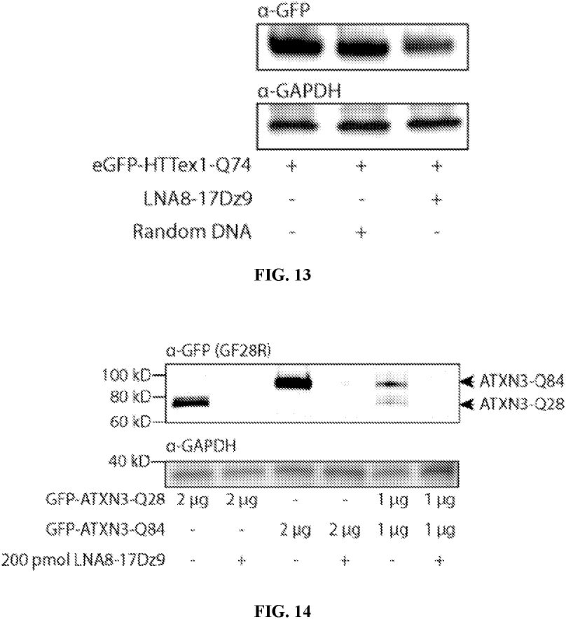

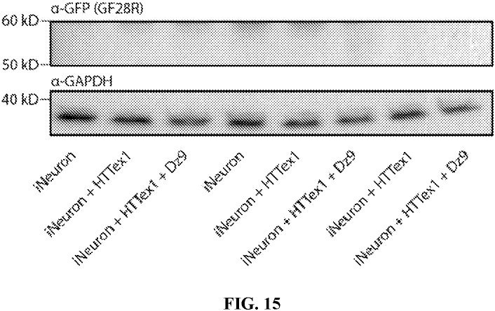

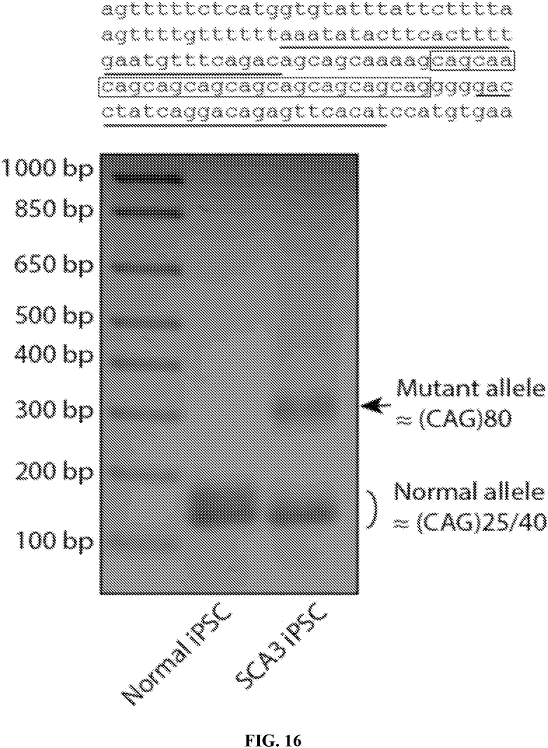

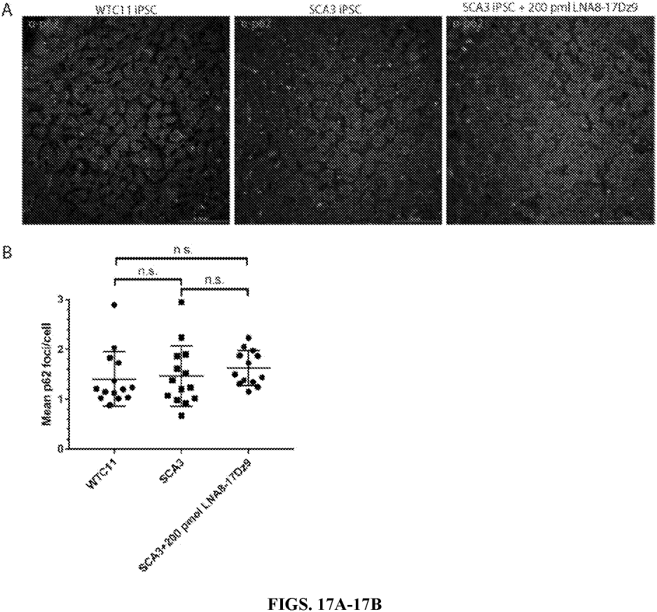

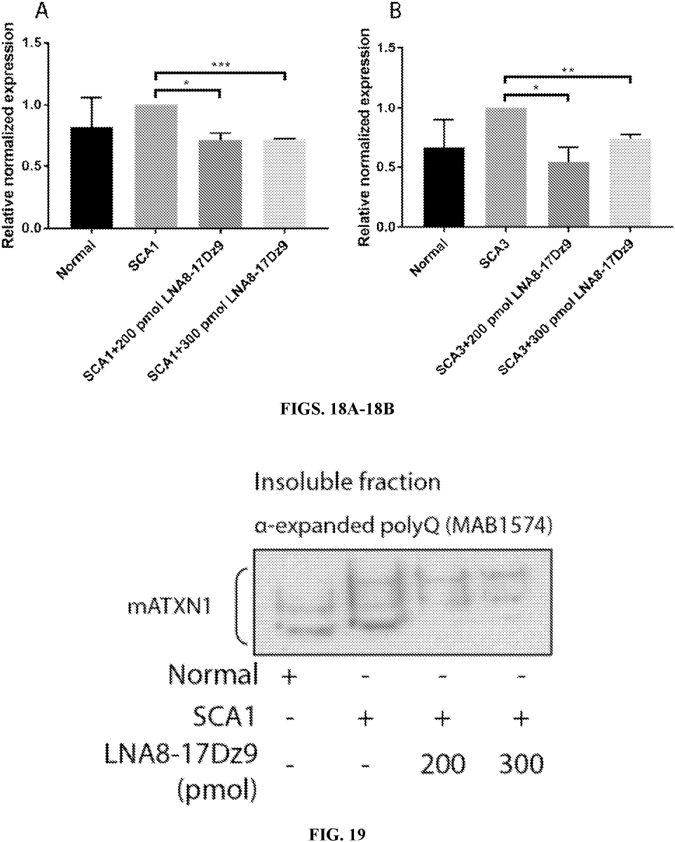

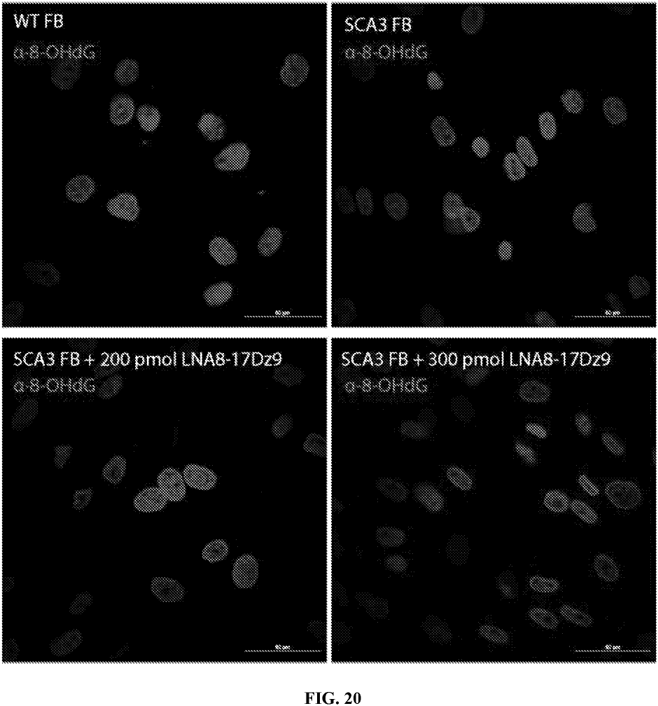

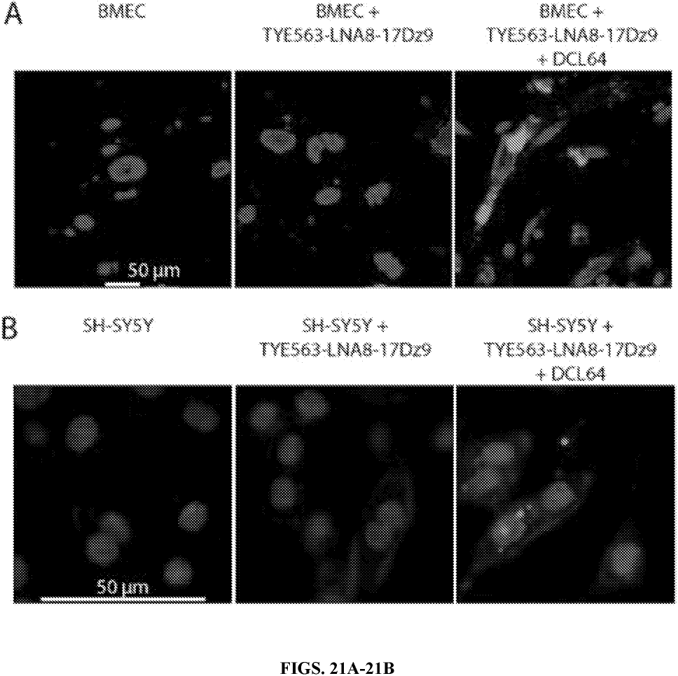

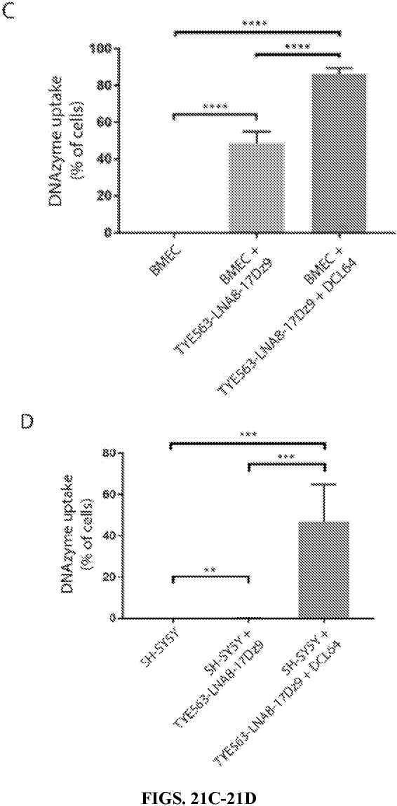

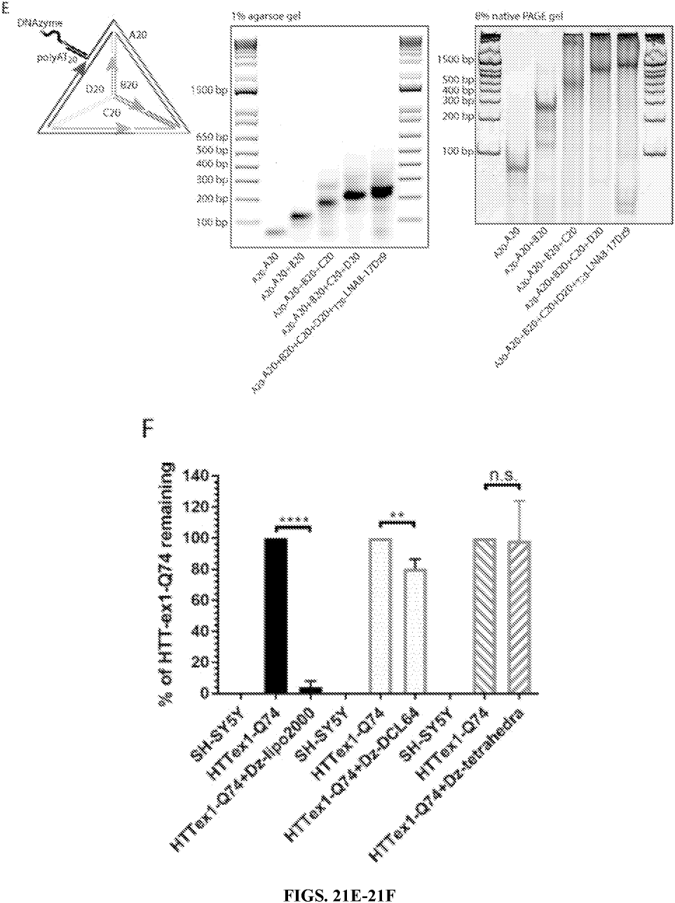

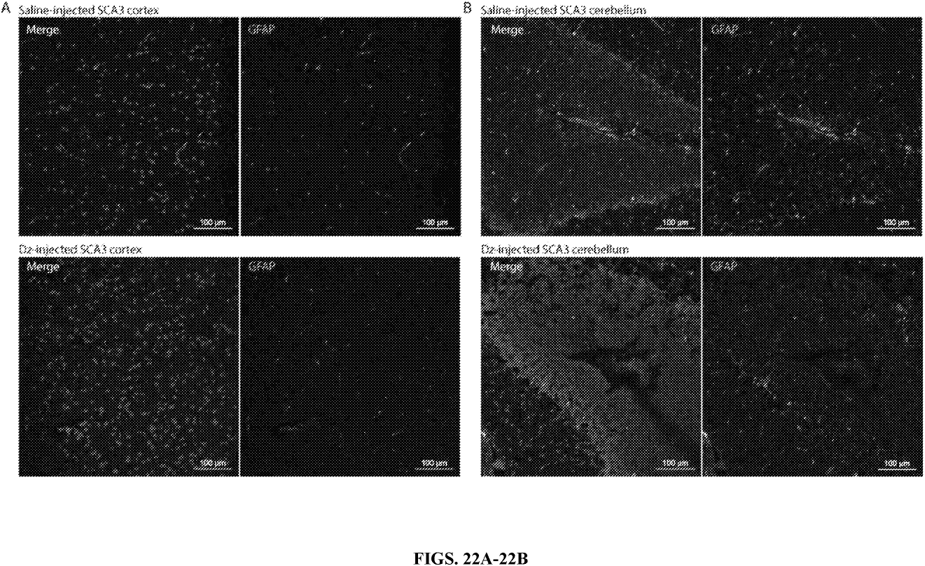

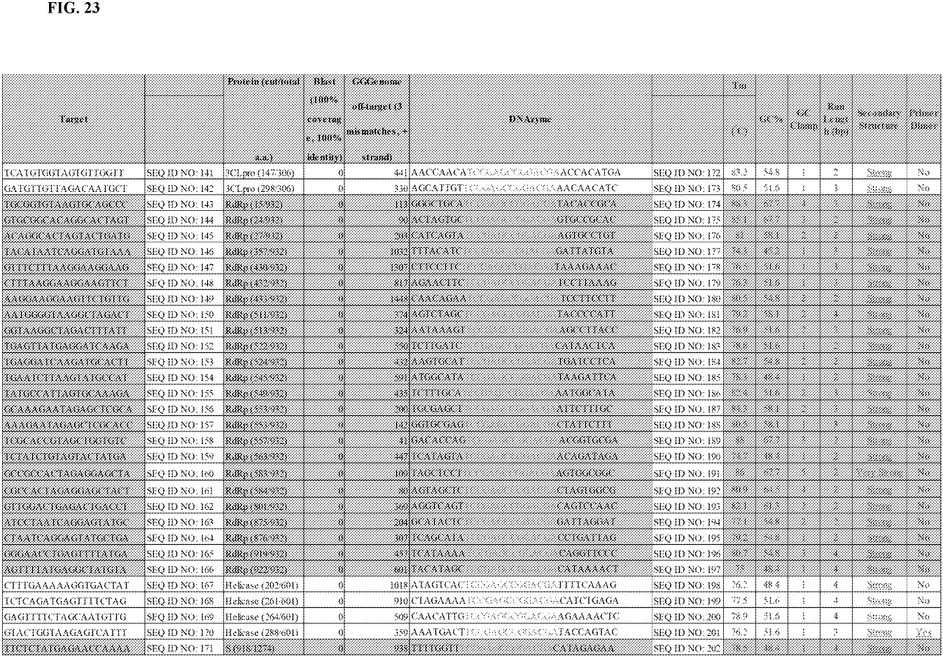

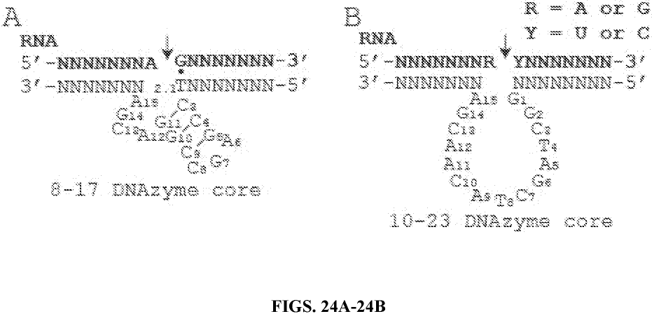

The T·G mismatch is indicated. FIG. 1B shows biochemical RNA cleavage assays at 37° C. for 1 h under different cation concentrations. A CTG probe complementary to the CAG RNA repeats were used as a negative control. The sequences in FIG. 1A include

CTGCTGCTGCTGTCCGAGCCGGACGAGCTGCTGCTGCTG (SEQ ID NO
: 129),

CAGCAGCAGCAGCAGCAGCAGCAGCAG (SEQ ID NO: 288),

+T+G+C+T+GCTGTCCGAGCCGGACGAGCTG+C+T+G+C+T (SEQ ID
NO: 130),

AGCAGCAGCAGCAGCAGCA (SEQ ID NO: 289),

*T*G*C*T*G*C*T*G*T*C*C*G*A*G*C*C*G*G*A*C*G*A*G*C*T
*G*C*T*G*C*T (SEQ ID NO: 131).

FIGS. 2A-2B show that DNAzyme effectively knocks down or eliminates an array of expanded polyQ proteins. FIG. 2A shows HTTex1-Q74 protein knockdown using three 8-17 Dz designs (200 pmol each, or 80 nM) in HEK293 cells. FIG. 2B shows that mutant ATXN1-Q83 (both bands were used for quantification), TBP-Q94, ATXN7-Q61 and AR-Q40 were significantly reduced or eliminated by 200 pmol (80 nM) LNA 8-17Dz9 in HEK293 cells.

FIGS. 3A-3E show that DNAzyme significantly reduces polyQ protein targets in neuronal cells and iPSCs. FIG. 3A shows that HTTex1-Q74 protein is significantly reduced in SH-SY5Y cells co- transfected with 200 pmol (80 nM) LNA8-17Dz9. FIG. 3B shows that iNeuron differentiation scheme and immunofluorescence (IF) of 5-day differentiated iNeurons against neuronal markers NeuN and MAP2 (with antibody clone numbers indicated in brackets, n = 3). FIG. 3C shows immunoblot and quantification of transfected HTTex1-Q74 levels with or without 200 pmol (80 nM) LNA8-17Dz9 treatment in 5-day differentiated iNeurons (n = 3). FIG. 3D shows IF of pluripotency markers in SCA3 iPSCs (n = 2). FIG. 3E shows immunoblot and quantification of mutant and wildtype ATXN3 in SCA3 iPSCs treated with 200 or 300 pmol (80 or 120 nM) LNA8- 17Dz9 (n = 4). The quantity of mutATXN3 in SCA3 iPSC (without treatment) was used as 100% for normalization.

FIG. 4 shows that allele specificity can be achieved by DNAzyme in SCA1 fibroblasts. Immunoblot and quantification of SCA1 fibroblasts nucleofected with 200 or 300 pmol LNA8-17Dz9 (n = 4). Protein bands used for quantification of mutant polyQ proteins are highlighted by the open bracket. FB - fibroblast.

FIG. 5 shows that DNAzyme knocks down both wt and mutATXN3 in SCA3 fibroblasts. Immunoblot and quantification of SCA3 fibroblasts nucleofected with 200 or 300 pmol LNA8-17Dz9 (n = 4). Protein bands used for quantification of mutant polyQ proteins are highlighted by the open bracket. FB - fibroblast.

FIGS. 6A-6B show that DNAzyme treatment rescues SCA3 fibroblast cell survival and had no detrimental effects on mitochondrial polarization. FIG. 6A shows cell proliferation assay of nucleofected SCA3 fibroblasts (200 or 300 pmol LNA8-17Dz9) in comparison to untreated cells and normal fibroblasts (2 biological replicates each with 4 technical replicates). FIG. 6B shows mitochondrial depolarization assay using JC-10 dye (2 biological replicates each with 4 technical replicates).

FIGS. 7A-7B show that DNAzyme reduces HMW ATXN3 in MJD84.2 mouse brain. FIG. 7A shows I.V.I.S. imaging of low-dosage (25 µg) Cy5-labeled LNA8-17Dz9 stereotaxically injected in a SCA3 right lateral ventricle. All images were normalized to the same fluorescent scale. FIG. 7B shows immunoblot and quantification of SCA3 mouse brain without treatment, with saline injection or with an intermediate dosage (400 µg) LNA8-17Dz9 (n = 3). The protein level of ATXN1 was not quantified due to promiscuity of the antibody.

FIG. 8 shows gels used for biochemical cleavage measurement in FIG. 1B. To establish a comparison to CAGₓ₃₀ cleavage efficiency, CAGₓ₄₂ was cloned into the pcDNA3.1 backbone for in vitro transcription. The size of the repeat is limited by the synthesis power from commercial companies.

FIG. 9 shows alternations of RNA binding arm’s length change the catalytic property of 8-17 Dz. Reactions were performed under different Mg²⁺ concentrations (mM) at 37° C. for 1 h using in vitro transcribed CAGₓ₃₀ RNA.

FIG. 10 shows that 8-17 Dz cleaves RNA target under low ionic conditions. Reactions were performed at 37° C. for 5 h using in vitro transcribed CAGₓ₃₀ RNA and 5 mM Mg²⁺.

FIG. 11 shows that LNA8-17Dz9 cleaves both CAGx30 and CAGx42 repeats with comparable efficiency biochemically. Reactions were performed at 37° C. for 1 h using in vitro transcribed CAGₓ₄₂ RNA and different concentrations of Mg²⁺. The CAGₓ₄₂ repeats were cloned into the pcDNA3.1 backbone for in vitro transcription. Generation of longer repeats is limited by the synthesis power commercially.

FIG. 12 shows HTTex1-Q74 mRNA expression after treatment with three 8-17Dz9 designs. Total RNA was extracted 48 h post transfection in HEK293 cells, reverse transcribed and used in qRT-PCR. RNA expression was normalized against GAPDH mRNA and expressed as fold change using Bio-Rad CFX Manager 3.1. Three biological replicates each with three technical replicates were used for calculation.

FIG. 13 shows that HTTex1-Q74 protein knockdown is not due to transfection competition from LNA8-17Dz9. Immunoblot of HEK293 cells co-transfected with GFP-HTTex1-Q74 and LNA8-17Dz9 or a random DNA fragment (of the same length) after 48 h.

FIG. 14 shows that LNA 8-17Dz9 cleaves both GFP-tagged ATXN3-Q28 and ATXN3-Q84 in HEK293 cells. HEK293 cells were transfected with 2 µg of ATXN3-Q28, 2 µg of ATXN3-Q84, or 1 µg of ATXN3-Q28 plus 1 µg of ATXN3-Q84 with or without 200 pmol LNA 8-17Dz9. Immunoblot was performed 48 h post transfection.

FIG. 15 shows that HTTex1-Q74 expression is completely lost on differentiation day 7 in iNeurons (n = 3).

FIG. 16 shows determination of CAG repeat size in a patient-derived SCA3 iPSC. PCR amplification of the ATXN3 repeat region (boxed sequence) was performed with primers (underlined sequence) and LongAmp (NEB) following the following thermo cycle: 95° C. 1 min , (95° C. 30 sec, 58° C. 30 sec, 65° C. 1 min) x 35, 65° C. 10 min, 4° C. hold. The sequence in FIG. 16 is

AGTTTTTCTCATGGTGTATTTATTCTTTTAAGTTTTGTTTTTTAAATATA
CTTCACTTTTGAATGTTTCAGACAGCAGCAAAAGCAGCAACAGCAGCAGC
AGCAGCAGCAGCAGGGGGACATATCAGGACAGAGTTCACATCCATGTGAA
(SEQ ID NO: 290).

FIGS. 17A-17B show that p62-dependent aggresome level is unaltered with DNAzyme treatment. FIG. 17A shows that IF against p62 in normal WTC11, untreated SCA3 and DNAzyme-treated SCA3 iPSCs (3 biological samples, 3266 cells were used for quantifying p62 aggresome in WTC11 iPSC, 3473 cells in untransfected SCA3 iPSC, and 2355 cells in transfected SCA3 iPSC). FIG. 17B is quantification of FIG. 17A.

FIGS. 18A-18B show qRT-PCR of target RNA knockdown by DNAzyme in SCA1 and SCA3 fibroblasts. FIG. 18A shows normalized ATXN1 RNA fold change in normal, untreated SCA1 and DNAzyme-treated SCA1 fibroblasts (2 biological samples each with 3 technical samples). FIG. 18B shows normalized ATXN3 RNA fold change in normal, untreated SCA3 and DNAzyme-treated SCA3 fibroblasts (2 biological samples each with 3 technical samples). The primers used for the qRT-PCR reactions were adopted from.

FIG. 19 shows that DNAzyme reduces mutATXN1 protein in the insoluble fraction of SCA1 fibroblast cells.

FIG. 20 shows that DNAzyme treatment does not change reactive oxygen species (ROS) induced DNA damage. IF was performed against 8-OHdG (n = 3).

FIGS. 21A-21F show packaging and delivery of LNA8-17Dz9 with liposome DCL64 or DNA tetrahedron. Retention of TYE563-LNA8-17Dz9 in BMECs with or without DCL64 packaging (FIGS. 21A and 21C, 2 biological replicates for each condition, 52 cells analyzed for BMEC+DNAzyme, 76 cells analyzed for BMEC+DNAzyme+DCL64). Retention of TYE563-LNA8-17Dz9 in SH-SY5Y cells with or without DCL64 packaging (FIGS. 21B and 21D, 2 biological replicates for each condition, >300 cells analyzed for SH-SY5Y+DNAzyme or SH-SY5Y+DNAzyme+DCL64). FIG. 21E shows that DNA tetrahedra with 20-nucleotide edge lengths (A-D20) were assembled and analyzed on agarose and native PAGE gels. The A20 strand has a poly A₂₀ overhang for hybridization with a poly T₂₀ tail on LNA8-17Dz9. FIG. 21F shows HTTex1-Q74 protein reduction by LNA8-17Dz9 delivered via lipofectamine 2000, liposome DCL64 or DNA tetrahedra (3 biological replicates for each condition).

FIGS. 22A-22B show IF imaging of the gliosis marker GFAP in saline-treated or DNAzyme-treated SCA3 mouse brain. (FIG. 22A) IF of cortex and (FIG. 22B) IF of cerebellum (One brain with 3 sections for each region examined).

FIG. 23 shows list of SARS-CoV/SARS-CoV-2 RNA targets and DNAzyme designs. Each viral target was first blasted on NCBI for off-target in the human genome. No off-targets with 100% identity 100% coverage were found for any of the viral target. Each viral target was then analyzed using GGGenome for off-target with mismatches (no more than 3 mismatches on the positive strand). The catalytic core of the DNAzyme is highlighted in blue and the chemical properties of each DNAzyme is evaluated using OligoEvaluator (Sigma-Aldrich). The catalytic core sequences in the DNAzyme column are shown as light grey.

FIGS. 24A-24B show a comparison of the 8-17 and 10-23 DNAzymes. Target RNA is highlighted in bold (N for nucleic acid). Cleavage sites are indicated by arrows: an AG cleavage site recognized by 8-17 Dz (FIG. 24A) and an RY cleavage site by 10-23 Dz (FIG. 24B). The sequence for the 8-17 DNAzyme core in FIG. 24A is

TCCGAGCCGGACGA (SEQ ID NO: 287).

The sequence for the 10-23 DNAzyme core in FIG. 24B is

GGCTAGCTACAACGA (SEQ ID NO: 291).

### DETAILED DESCRIPTION

DNAzymes are DNA molecules that specifically recognize and cleave a polynucleotide in a sequence-specific manner. A major challenge lies in the design of a functional DNAzyme that targets CAG repeats. Further, a DNAzyme that targets highly conserved regions of a polynucleotide of a pathogen (e.g., a virus) is needed. Accordingly, disclosed herein are DNAzyme compositions and methods for cleaving nucleic acids and treating polyglutamine diseases and viral diseases.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98% to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

The term “DNAzyme” as used herein means a DNA molecule that specifically recognizes and cleaves a polynucleotide in a sequence-specific manner. A DNAzyme normally contains a catalytic core that is responsible for catalysis and two substrate-binding arms of variable length and sequence. The substrate-binding arms bind the target polynucleotide in a sequence-specific manner. It will be appreciated by those skilled in the art, however, that strict complementarity may not be required for the DNAzyme to bind to and cleave the target nucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs, or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

“Therapeutically effective amount” or “therapeutically effective dose” of a composition refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The phrases “concurrent administration”, “administration in combination”, “simultaneous administration” or “administered simultaneously” as used herein, means that the compounds are administered at the same point in time or immediately following one another.

The term “biocompatible” generally refers to a material and any metabolites or degradation products thereof that are generally non-toxic to the recipient and do not cause significant adverse effects to the subject

The term “polypeptide” refers to a compound made up of a single chain of D- or L-amino acids or a mixture of D- and L-amino acids joined by peptide bonds.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Engineered Nucleic Acids

Disclosed herein are engineered nucleic acids that target CAG repeat sequences or viral polynucleotides.

In some aspects, disclosed herein is an engineered nucleic acid comprising:
a first binding arm,
a catalytic core domain, and
a second binding arm,
wherein the catalytic core domain is in between the first binding arm and the second binding arm, and wherein the first binding arm and the second binding arm target a CAG repeat sequence.

The term “CAG repeat sequence” refers to a polynucleotide sequence comprising at least 3 continuous repeats of CAG (including, for example, at least 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 repeats of CAG.

In some embodiments, the catalytic core domain is an 8-17 catalytic core. In some embodiments, the catalytic core domain comprises a sequence at least about 80% (for example, at least about 80%, 85%, 90%, 92%, 93%, 94%, 95%, 96%, 97%, 98%, or 99%) identical to SEQ ID NO: 287. In some embodiments, the catalytic core domain comprises the sequence SEQ ID NO: 287.

In some embodiments, the catalytic core domain is a 10-23 catalytic core. In some embodiments, the catalytic core domain comprises a sequence at least about 80% (for example, at least about 80%, 85%, 90%, 92%, 93%, 94%, 95%, 96%, 97%, 98%, or 99%) identical to SEQ ID NO: 291. In some embodiments, the catalytic core domain comprises the sequence SEQ ID NO: 291.

In some embodiments, the first binding arm and the second binding arm comprise at least 3 nucleotides (for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides). In some embodiments, the first and the second binding arms comprise 6, 7, 8, 9, 10, 11, 12, or 13 nucleotides. In some embodiments, the first binding arm and the second binding arm comprise 9 nucleotides.

The first binding arm and the second binding arm of the engineered nucleic acid can target the CAG repeat sequence. In some embodiments, the first binding arm and the second binding arm of the engineered nucleic acid is at least about at least about 90% (for example, at least about 90%, 91%, 92%, 93%, 94%, 95%, 96%, 97%, 98%, or 99%) complementary to the CAG repeat sequence.

In some embodiments, the first binding arm and the second binding arm comprise a sequence at least 80% (for example, at least about 80%, 85%, 90%, 92%, 93%, 94%, 95%, 96%, 97%, 98%, or 99%) identical to SEQ ID NOs: 265-286. In some embodiments, the first binding arm and the second binding arm comprise the sequence selected from SEQ ID NO: 265-286.

In some embodiments, the first binding arm comprises a sequence at least 80% (for example, at least about 80%, 85%, 90%, 92%, 93%, 94%, 95%, 96%, 97%, 98%, or 99%) identical to SEQ ID NO: 265, 267, 269, 271, 273, 275, 277, 279, 281, 283, or 285. In some embodiments, the first binding arm comprises the sequence selected from SEQ ID NOs: 265, 267, 269, 271, 273, 275, 277, 279, 281, 283, and 285.

In some embodiments, the second binding arm comprises a sequence at least 80% (for example, at least about 80%, 85%, 90%, 92%, 93%, 94%, 95%, 96%, 97%, 98%, or 99%) identical to SEQ ID NO: 266, 268, 270, 272, 274, 276, 278, 280, 282, 284, or 286. In some embodiments, the second binding arm comprises the sequence selected from SEQ ID NOs: 266, 268, 270, 272, 274, 276, 278, 280, 282, 284, and 286.

Also disclosed herein is an engineered nucleic acid comprising:
a first binding arm,
a catalytic core domain, and
a second binding arm,
wherein the catalytic core domain is in between the first binding arm and the second binding arm, and wherein the first binding arm and the second binding arm target a viral polynucleotide.

In some embodiments, the first binding arm and the second binding arm are complementary to a viral polynucleotide. The viral polynucleotide can be a polynucleotide of a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some embodiments, the viral polynucleotide is a coronavirus polynucleotide. In some embodiments, the viral polynucleotide is a SARS-CoV-2 polynucleotide (e.g., an RNA).

In some examples, the first binding arm and the second binding arm of the engineered nucleic acid disclosed herein can target a conserved region of a SARS-CoV-2 polynucleotide. In some embodiments, the first binding arm and the second binding arm of the engineered nucleic acid is at least about at least about 90% (for example, at least about 90%, 91%, 92%, 93%, 94%, 95%, 96%, 97%, 98%, or 99%) complementary to the SARS-CoV-2 polynucleotide. In some embodiments, the SARS-CoV-2 polynucleotide is selected from SEQ ID NOs: 141-171.

In some embodiments, the first binding arm and the second binding arm comprise a sequence at least 80% (for example, at least about 80%, 85%, 90%, 92%, 93%, 94%, 95%, 96%, 97%, 98%, or 99%) identical to SEQ ID NOs: 203-264. In some embodiments, the first binding arm and the second binding arm comprise the sequence selected from SEQ ID NOs: 203-264.

In some embodiments, the first binding arm comprises a sequence at least 80% (for example, at least about 80%, 85%, 90%, 92%, 93%, 94%, 95%, 96%, 97%, 98%, or 99%) identical to SEQ ID NOs: 203-233. In some embodiments, the first binding arm comprises the sequence selected from SEQ ID NOs: 203-233.

In some embodiments, the second binding arm comprises a sequence at least 80% (for example, at least about 80%, 85%, 90%, 92%, 93%, 94%, 95%, 96%, 97%, 98%, or 99%) identical to SEQ ID NOs: 234-264. In some embodiments, the second binding arm comprises the sequence selected from SEQ ID NOs: 234-264.

In some embodiments, at least one nucleotide of the engineered nucleic acid is a chemically modified ribose. In some embodiments, at least one nucleotide at the 5′-terminus and at least one nucleotide at the 3′-termius of the engineered nucleic acid are chemically modified. In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, locked nucleic acid (LAN), peptide nucleic acid (PNA), Methylene-cLAN, N-MeO-amino BNA, or N-MeO-aminooxy BNA. In one embodiment, the chemically modified ribose is a locked nucleic acid (LAN). In one embodiment, a peptide nucleic acid (PNA).

The structures of these modified riboses are shown below:

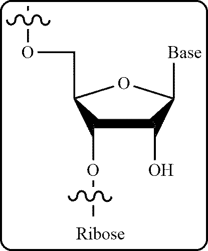

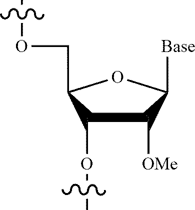

2′-O-methyl (2′-O-Me)

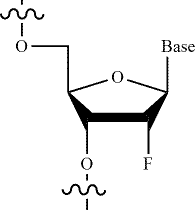

2′-Fluoro (2′-F)

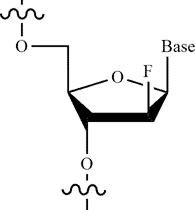

2′-deoxy-2′-fluoro-beta-D-arabino-nucleic acid (2′F-ANA)

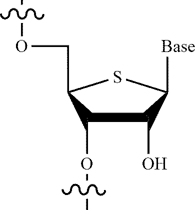

4′-S

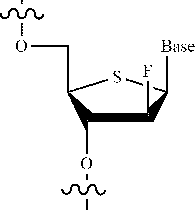

4′-SFANA

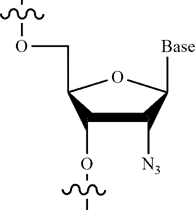

2′-azido

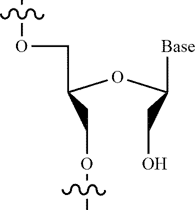

UNA

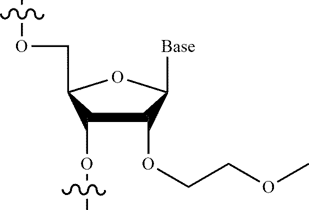

2′-O-methoxy-ethyl (2′-O-ME)

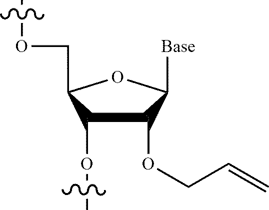

2′-O-Allyl

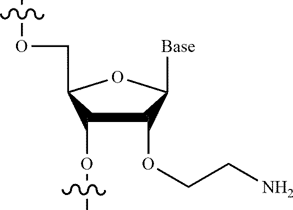

2′-O-Ethylamine

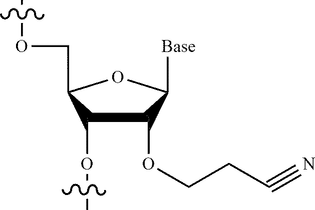

2′-O-Ethylamine

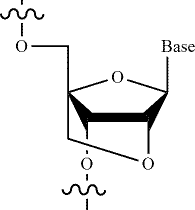

Locked nucleic acid (LAN)

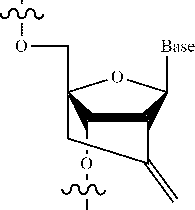

Methylene-cLAN

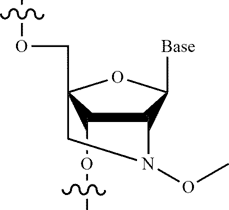

N-MeO-amino BNA

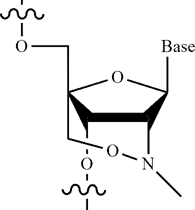

N-MeO-aminooxyBNA

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

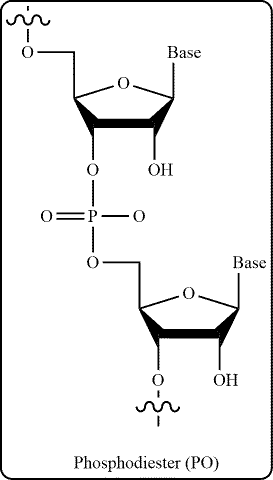

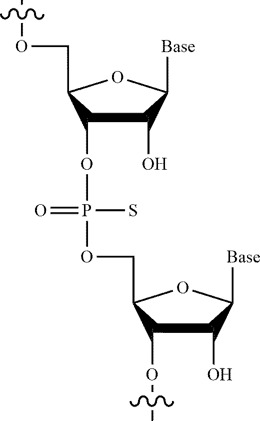

Phosphorothioate (PS)

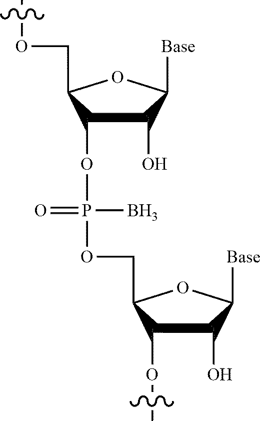

Boranophosphate

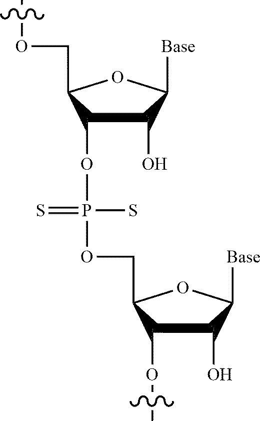

Phosphodithioate(PS2)

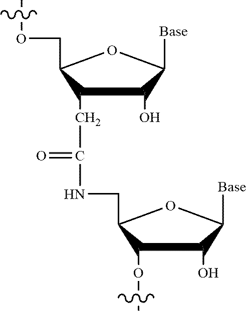

3′, 5′ -amide

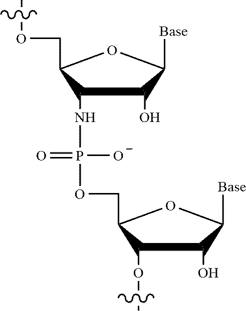

N3′-phosphoramidate (NP)

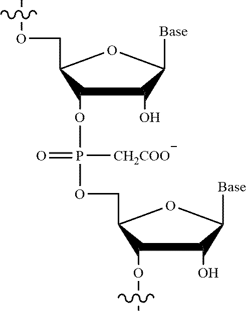

Phosphodiester (PO)

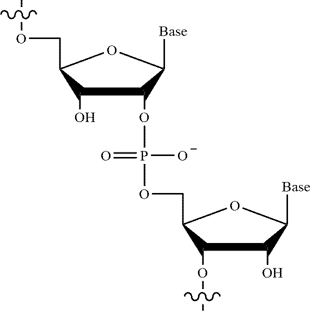

2′,5′-phosphodiester (2′,5′-PO)

In some embodiments, the engineered nucleic acid targeting CAG repeat sequence comprises a sequence at least 80% (for example, at least about 80%, 85%, 90%, 92%, 93%, 94%, 95%, 96%, 97%, 98%, or 99%) identical to SEQ ID NOs: 121-132.

In some embodiments, the engineered nucleic acid targeting SARS-CoV-2 polynucleotide sequence comprises a sequence at least 80% (for example, at least about 80%, 85%, 90%, 92%, 93%, 94%, 95%, 96%, 97%, 98%, or 99%) identical to SEQ ID NOs: 172-202

The engineered nucleic acid disclosed herein can be any metal-assisted DNAzyme (for example, Pb²⁺, Mn²⁺, Mg²⁺, Zn²⁺, or Ca²⁺). In some embodiments, the DNAzyme cleaves the target RNA in the presence of Ca²⁺. In some embodiments, the DNAzyme cleaves the target RNA in the presence of Mg²⁺. In some embodiments, the DNAzyme cleaves the target RNA in the presence of Zn²⁺. In some embodiments, the DNAzyme cleaves the target RNA in the presence of Pb²⁺. In some embodiments, the DNAzyme cleaves the target RNA in the presence of Mn²⁺.

In some aspects, disclosed herein is a pharmaceutical composition comprising the engineered nucleic acid disclosed herein and a pharmaceutically acceptable carrier.

In some aspects, disclosed herein is an engineered nucleic acid, comprising:
a first binding arm,
a catalytic core domain, and
a second binding arm,
wherein the catalytic core domain is in between the first binding arm and the second binding arm, and wherein the first binding arm and the second binding arm specifically bind to a CAG repeat RNA sequence.

In some aspects, disclosed herein is an engineered nucleic acid, comprising:
a first binding arm,
a catalytic core domain, and
a second binding arm,
wherein the catalytic core domain is in between the first binding arm and the second binding arm, and wherein the first binding arm and the second binding arm specifically bind to a SARS-CoV-2 RNA sequence. Also disclosed herein is a method for cleaving a CAG repeat RNA sequence comprising the step of contacting the CAG repeat RNA sequence with an engineered nucleic acid under conditions suitable for the cleavage of the CAG repeat RNA sequence by the engineered nucleic acid, wherein the engineered nucleic acid comprises:
a first binding arm,
a catalytic core domain, and
a second binding arm,
wherein the catalytic core domain is in between the first binding arm and the second binding arm, and wherein the first binding arm and the second binding arm specifically bind to a CAG repeat RNA sequence.

Further disclosed herein is a method for cleaving a SARS-CoV-2 RNA sequence comprising the step of contacting the SARS-CoV-2 RNA sequence with an engineered nucleic acid under conditions suitable for the cleavage of the SARS-CoV-2 RNA sequence by the engineered nucleic acid, wherein the engineered nucleic acid comprises:
a first binding arm,
a catalytic core domain, and
a second binding arm,
wherein the catalytic core domain is in between the first binding arm and the second binding arm, and wherein the first binding arm and the second binding arm specifically bind to a SARS-CoV-2 RNA sequence.

### Methods for Treating Polyglutamine Diseases

The disclosed invention targets CAG repeats on an RNA level rather than a DNA level (as opposed to CRISPR-Cas) and can be much safer without inducing permanent genomic damage. DNAzymes are also more stable than RNA-based therapies and can be further stabilized through chemical modifications.

Accordingly, also disclosed herein are methods of preventing, inhibiting, reducing, and/or treating a polyglutamine disease in subject in need thereof comprising administering to the subject in need thereof a therapeutically effective amount of the engineered nucleic acid disclosed herein.

It should be understood herein that the term “polyglutamine disease” refers to a group of neurodegenerative disorders caused by an increased numbers of CAG repeats that encode glutamine. The increased numbers of CAG repeats can occur throughout all genomes, within a gene, within one or more coding regions of a gene, or within an RNA. In some embodiments, the number of the CAG repeats in the subject is at least about 1.5 times (for example, at least about 1.5 times, at least about 1.6 times, at least about 1.7 times, at least about 1.8 times, at least about 2 times, at least about 3 times, at least about 4 time, at least about 5 times, at least about 6 times, at least about 7 times, at least about 8 times, at least about 9 times, at least about 10 times, at least about 15 times, at least about 20 times, at least about 25 times, at least about 30 times, at least about 40 times, at least about 50 times, at least about 100 times, at least about 150 times, at least about 200 times, at least about 300 times, at least about 400 times, at least about 500 times, or at least about 1000 times) more than a reference control. The term “reference control” refers to a level in detected in a subject in general or a study population (e.g., healthy control).

As discussed herein, the engineered nucleic acid can target CAG repeat sequences. In some embodiments, the subject comprises a polyglutamine disease-related polynucleotide comprising an increased level of CAG repeat sequence as compared to a reference control. Accordingly, in some embodiments, the engineered nucleic acid decreases a level of a polyglutamine disease-related polynucleotide.

The polyglutamine disease-related polynucleotide disclosed herein includes, for example, a huntingtin (HTT) polynucleotide, an ATXN1 polynucleotide, an ATXN2 polynucleotide, an ATXN7 polynucleotide, a TATA binding protein-coding polynucleotide, an androgen receptor (AR) polynucleotide, or an atrophin 1 (ATN1) polynucleotide.

Huntingtin (HTT) is a disease gene linked to Huntington’s disease. “Huntingtin protein” or “Huntingtin polypeptide” refers herein to a polypeptide that synthesizes and hydrolyzes cyclic adenosine 5′-diphosphate-ribose, and in humans, is encoded by the HTT gene. In some embodiments, the HTT polynucleotide is that identified in one or more publicly available databases as follows: NC_000004.12, NM_002111.8, AB016794.1, AB209506.1, AK025918.1, AK290544.1, BC014028.2, BM661887.1, KJ535072.1, L12392.1, or L20431.1.

ATXN1 (Ataxin 1) is a diseases gene associated with spinocerebellar ataxia type 1 (SCA1) and hereditary ataxia. “ATXN1 protein” or “ATXN1 polypeptide” refers herein to a polypeptide that synthesizes and hydrolyzes cyclic adenosine 5′-diphosphate-ribose, and in humans, is encoded by the ATXN1 gene. In some embodiments, the ATXN1 polynucleotide is that identified in one or more publicly available databases as follows: NC_000006.12, NM_000332.3, NM_001128164.2, or NM_001357857.2.

ATXN2 (Ataxin 2) is a diseases gene associated with spinocerebellar ataxia type 2 (SCA2) and Parkinson’s disease. “ATXN2 protein” or “ATXN2 polypeptide” refers herein to a polypeptide that synthesizes and hydrolyzes cyclic adenosine 5′-diphosphate-ribose, and in humans, is encoded by the ATXN2 gene. In some embodiments, the ATXN2 polynucleotide is that identified in one or more publicly available databases as follows: NC_000012.12, NM_001310121.1, NM_001310123.1, NM_001372574.1, or NM_002973.4.

ATXN7 (Ataxin 7) is a diseases gene associated with Spinocerebellar Ataxia type 7 (SCA7) and retinal degeneration. “ATXN7 protein” or “ATXN7 polypeptide” refers herein to a polypeptide that synthesizes and hydrolyzes cyclic adenosine 5′-diphosphate-ribose, and in humans, is encoded by the ATXN7 gene. In some embodiments, the ATXN7 polynucleotide is that identified in one or more publicly available databases as follows: NM_000333.4, NM_001128149.3, NM_001177387.1, NM_001377405.1, or NM_001377406.1.

TATA-Box binding protein (TBP) is associated with spinocerebellar ataxia 17 and Parkinson’s disease. “TBP” refers herein to a polypeptide that synthesizes and hydrolyzes cyclic adenosine 5′-diphosphate-ribose, and in humans, is encoded by the TBP gene. In some embodiments, the TBP polynucleotide is that identified in one or more publicly available databases as follows: NC_000006.12, NM_001172085.1, or NM_003194.5.

Androgen receptor (AR) is associated with spinobulbar muscular atrophy (SBMA). “AR” refers herein to a polypeptide that synthesizes and hydrolyzes cyclic adenosine 5′-diphosphate-ribose, and in humans, is encoded by the AR gene. In some embodiments, the AR polynucleotide is that identified in one or more publicly available databases as follows: NC_000023.11, NM_000044.6, NM_001011645.3, NM _001348061.1, NM_001348063.1, or NM_001348064.1.

ATN1 (atrophin 1) is a diseases gene associated with Dentatorubral-pallidoluysian atrophy (DRPLA). “ATN1 protein” or “ATN1 polypeptide” refers herein to a polypeptide that synthesizes and hydrolyzes cyclic adenosine 5′-diphosphate-ribose, and in humans, is encoded by the ATN1 gene. In some embodiments, the ATN1 polynucleotide is that identified in one or more publicly available databases as follows: NC_000012.12, NM_001007026.2, NM_001940.4.

The methods and compositions disclosed herein are effective on decreasing polyglutamine disease-related polynucleotide. Accordingly, in some embodiments, the methods and compositions disclosed herein are effective on treating/preventing/mitigating a polyglutamine disease selected from Huntington’s disease (HD), spinocerebellar ataxias (SCA) type 1, SCA type 2, SCA type 3, SCA type 6, SCA type 7, SCA type 17, Dentatorubral-pallidoluysian atrophy (DRPLA), spinobulbar muscular atrophy (SBMA), hereditary ataxia, Parkinson’s disease, and retinal degeneration. In some embodiments, the methods and compositions disclosed herein are effective on treating/preventing/mitigating a polyglutamine disease selected from Huntington’s disease (HD), spinocerebellar ataxias (SCA) type 1, SCA type 2, SCA type 3, SCA type 6, SCA type 7, SCA type 17, Dentatorubral-pallidoluysian atrophy (DRPLA), and spinobulbar muscular atrophy (SBMA).

The CAG-repeat based DNAzyme can also target antisense CAG RNA (or RNA foci) arising from sense CUG repeat expansions, as many repeat expansions are bi-directionally transcribed. Examples of CUG expansion diseases include myotonic dystrophy type 1, Fuchs endothelial corneal dystrophy (FECD), Huntington disease like 2 (HDL2) and SCA8. The antisense CAG RNA/foci can contribute to pathology by sequestering RNA binding proteins in the nucleus/cytoplasm (RNA gain-of-function) or by initiating repeat-associated non-AUG (RNA) translation after being transported to the cytoplasm.

In addition, by changing the binding arm specificity, unique sequences can be targeted in addition to CAG repeats. Thus, the DNAzyme technology can be applied to a disease with a toxic RNA component/etiology. The combination of 8-17 and/or 10-23 DNAzymes can cover almost all repeat types from microsatellite expansion diseases.

“Huntington’s Disease” or “HD” herein refers to an inherited autosomal dominant genetic disorder caused by expansions of CAG repeats (polyglutamine-polyQ) at the N-terminus, within, for example, exon 1, of the HTT protein. These polyglutamine expansions are highly associated with cytotoxicity and aggregates formation. HD is marked by neuronal tissue degeneration and appears be due to the development of protein aggregates that arise initially from the misfolding of the mutant HTT protein. It should be understood that a treatment of HD may be a treatment of one or more of movement, thinking (cognitive), and/or psychiatric disorders, and/or shown as a decrease in the level of CAG repeat, disease-associated polynucleotides/polypeptides, or a slower formation of aggregates.

Spinocerebellar ataxia (SCA) is a term referring to a group of hereditary ataxias that are characterized by degenerative changes in the part of the brain related to the movement control (cerebellum), and sometimes in the spinal cord. There are many different types of SCA, and they are classified according to the mutated (altered) gene responsible for the specific type of SCA. The types are described using “SCA” followed by a number, according to their order of identification: SCA1 through SCA40 (and the number continues to grow). The signs and symptoms may vary by type but are similar, and may include an uncoordinated walk (gait), poor hand-eye coordination, and abnormal speech (dysarthria). It should be understood that a treatment of spinocerebellar ataxia may be a treatment of one or more of uncoordinated walk (gait), poor hand-eye coordination, and/or abnormal speech (dysarthria), and/or shown as a decrease in the level of CAG repeat or disease-associated polynucleotides/polypeptides.

Dentatorubral-pallidoluysian atrophy (DRPLA) is an autosomal dominant neurodegenerative disorder characterized by ataxia, choreoathetosis, dementia, and psychiatric disturbance in adults and ataxia, myoclonus, seizures, and progressive intellectual deterioration. Characteristic neuropathologic observations include degeneration of the dentatorubral and pallidoluysian systems of the central nervous system. It should be understood that a treatment of Dentatorubral-pallidoluysian atrophy may be a treatment of one or more of degeneration of the dentatorubral and pallidoluysian systems of the central nervous system, and/or shown as a decrease in the level of CAG repeat or disease-associated polynucleotides/polypeptides.

“X-linked spinal and bulbar muscular atrophy” (or spinobulbar muscular atrophy: SBMA; or Kennedy disease) is characterized by onset of progressive muscle weakness, atrophy, and fasciculations typically in the fourth or fifth decade of life. SBMA is caused by an expansion of the CAG trinucleotide repeat in, for example, exon 1 of the human androgen receptor (AR) gene. It should be understood that a treatment of spinocerebellar ataxia may be a treatment of one or more of muscle weakness, atrophy, and fasciculations, and/or shown as a decrease in the level of CAG repeat or disease-associated polynucleotides/polypeptides.

In some embodiments, the catalytic core domain is an 8-17 catalytic core or a 10-23 catalytic core. In some embodiments, the catalytic core domain comprises a sequence at least about 80% (for example, at least about 80%, 85%, 90%, 92%, 93%, 94%, 95%, 96%, 97%, 98%, or 99%) identical to SEQ ID NO: 287. In some embodiments, the catalytic core domain comprises the sequence SEQ ID NO: 287.

In some embodiments, the catalytic core domain is a 10-23 catalytic core. In some embodiments, the catalytic core domain comprises a sequence at least about 80% (for example, at least about 80%, 85%, 90%, 92%, 93%, 94%, 95%, 96%, 97%, 98%, or 99%) identical to SEQ ID NO: 291. In some embodiments, the catalytic core domain comprises the sequence SEQ ID NO: 291.

In some embodiments, the first binding arm and the second binding arm comprise at least 3 nucleotides (for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides). In some embodiments, the first and the second binding arms comprise 6, 7, 8, 9, 10, 11, 12, or 13 nucleotides. In some embodiments, the first binding arm and the second binding arm comprise 9 nucleotides.

The first binding arm and the second binding arm of the engineered nucleic acid can target the CAG repeat sequence. In some embodiments, the first binding arm and the second binding arm of the engineered nucleic acid is at least about at least about 90% (for example, at least about 90%, 91%, 92%, 93%, 94%, 95%, 96%, 97%, 98%, or 99%) complementary to the CAG repeat sequence.

In some embodiments, the first binding arm and the second binding arm comprise a sequence at least 80% (for example, at least about 80%, 85%, 90%, 92%, 93%, 94%, 95%, 96%, 97%, 98%, or 99%) identical to SEQ ID NOs: 265-286. In some embodiments, the first binding arm and the second binding arm comprise the sequence selected from SEQ ID NO: 265-286.

In some embodiments, the first binding arm comprises a sequence at least 80% (for example, at least about 80%, 85%, 90%, 92%, 93%, 94%, 95%, 96%, 97%, 98%, or 99%) identical to SEQ ID NO: 265, 267, 269, 271, 273, 275, 277, 279, 281, 283, or 285. In some embodiments, the first binding arm comprises the sequence selected from SEQ ID NOs: 265, 267, 269, 271, 273, 275, 277, 279, 281, 283, and 285.

In some embodiments, the second binding arm comprises a sequence at least 80% (for example, at least about 80%, 85%, 90%, 92%, 93%, 94%, 95%, 96%, 97%, 98%, or 99%) identical to SEQ ID NO: 266, 268, 270, 272, 274, 276, 278, 280, 282, 284, or 286. In some embodiments, the second binding arm comprises the sequence selected from SEQ ID NOs: 266, 268, 270, 272, 274, 276, 278, 280, 282, 284, and 286.

In some embodiments, at least one nucleotide of the engineered nucleic acid is a chemically modified ribose. In some embodiments, at least one nucleotide at the 5′-terminus and at least one nucleotide at the 3′-termius of the engineered nucleic acid are chemically modified. In one embodiment, the chemically modified ribose is selected from 2′-O-methyl (2′- O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, locked nucleic acid (LAN), peptide nucleic acid (PNA), Methylene-cLAN, N-MeO-amino BNA, or N-MeO-aminooxy BNA. In one embodiment, the chemically modified ribose is a locked nucleic acid (LAN). In one embodiment, a peptide nucleic acid (PNA).

In one embodiment, the at least one chemically modified nucleotide is a chemically modified phosphodiester linkage.

In some embodiments, the engineered nucleic acid comprises a sequence at least 80% identical to SEQ ID NOs: 121-132.

The engineered nucleic acid disclosed herein can be any metal-assisted DNAzyme (for example, Pb²⁺, Mn²⁺, Mg²⁺, Zn²⁺, or Ca²⁺).

In some embodiments, the method comprises administering to the subject a therapeutically effective amount of a pharmaceutical composition comprising the engineered nucleic acid disclosed herein and a pharmaceutically acceptable carrier.

In some embodiments, the composition described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

As the timing of a disease can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### Methods for Treating a Viral Infection

Also disclosed herein are methods of preventing, treating, reducing, and/or inhibiting a viral infection in subject in need thereof comprising administering to the subject in need thereof a therapeutically effective amount of the engineered nucleic acid disclosed herein.

In some embodiments, a viral infection is an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In some embodiments, the infection is a coronavirus infection. In some embodiments, the infection is s SARS-CoV-2 infection.

In some aspects, disclosed herein are methods of preventing, treating, reducing, and/or inhibiting a SARS-CoV-2 infection in subject in need thereof comprising administering to the subject in need thereof a therapeutically effective amount of the engineered nucleic acid disclosed herein.

In some examples, the first binding arm and the second binding arm of the engineered nucleic acid disclosed herein can target a conserved region of a SARS-CoV-2 polynucleotide. In some embodiments, the first binding arm and the second binding arm of the engineered nucleic acid is at least about at least about 90% (for example, at least about 90%, 91%, 92%, 93%, 94%, 95%, 96%, 97%, 98%, or 99%) complementary to the SARS-CoV-2 polynucleotide. In some embodiments, the SARS-CoV-2 polynucleotide is selected from SEQ ID NOs: 1-120. In some embodiments, the first binding arm and the second binding arm of the engineered nucleic acid is at least about at least about 90% (for example, at least about 90%, 91%, 92%, 93%, 94%, 95%, 96%, 97%, 98%, or 99%) complementary to the SARS-CoV-2 polynucleotide. In some embodiments, the SARS-CoV-2 polynucleotide is selected from SEQ ID NOs: 141-171.

In some embodiments, the first binding arm and the second binding arm comprise a sequence at least 80% (for example, at least about 80%, 85%, 90%, 92%, 93%, 94%, 95%, 96%, 97%, 98%, or 99%) identical to SEQ ID NOs: 203-264. In some embodiments, the first binding arm and the second binding arm comprise the sequence selected from SEQ ID NOs: 203-264.

In some embodiments, the first binding arm comprises a sequence at least 80% (for example, at least about 80%, 85%, 90%, 92%, 93%, 94%, 95%, 96%, 97%, 98%, or 99%) identical to SEQ ID NOs: 203-233. In some embodiments, the first binding arm comprises the sequence selected from SEQ ID NOs: 203-233.

In some embodiments, the second binding arm comprises a sequence at least 80% (for example, at least about 80%, 85%, 90%, 92%, 93%, 94%, 95%, 96%, 97%, 98%, or 99%) identical to SEQ ID NOs: 234-264. In some embodiments, the second binding arm comprises the sequence selected from SEQ ID NOs: 234-264.

In some embodiments, the method comprises administering to the subject a therapeutically effective amount of a pharmaceutical composition comprising the engineered nucleic acid disclosed herein and a pharmaceutically acceptable carrier.

In some embodiments, the composition described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

The disclosed methods of treating, preventing, reducing, and/or inhibiting the infection described herein can be used prior to or following the onset of the infection, to treat, prevent, inhibit, and/or reduce the infection or symptoms thereof. In one aspect, the disclosed methods can be employed 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to the infection; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months after the infection.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. DNAzyme Cleavage of CAG Repeat RNA in Polyglutamine Diseases

DNAzymes (Dzs) are single-stranded RNA-cleaving DNAs that typically consist of a catalytic core and two flanking RNA binding arms for target recognition. The catalytic core cleaves a phosphodiester bond in the presence of divalent cations, generating a 5′- product with a 2′, 3′-cyclic phosphate at the 3′-end and a 3′-product with a hydroxyl groupat the 5′-end. The 8-17 DNAzyme is amongst the first identified DNAzymes that efficiently cleave RNA substrates. The 15-nucleotide (nt) core has been independently isolatedunder different cation selections such as Mg²⁺, Mn²⁺, Ca²⁺, Zn²⁺ and Pb²⁺ and shown to twist into a pseudoknot with the two RNA binding arms 70 ° apart in the crystal structure. Despite their high reprogrammability and versatility for chemical modification and nanoconjugation, the theranostic value and means of delivery are largely unexplored.

Polyglutamine (polyQ) diseases are a group of incurable neurodegenerative disorders caused by CAG repeat expansion in coding regions, including Huntington’s disease (HD), spinocerebellar ataxias (SCA types 1, 2, 3, 6, 7 and 17), dentatorubropallidoluysian atrophy (DRPLA), and spinobulbar muscular atrophy (SBMA). A toxic gain-of-function of the expanded polyQ proteins has been proposed to cause a variety of potential pathogenic cellular events, including single- or double-stranded (ss/ds) DNA breaks, impaired protein homeostasis, transcription dysregulation, protein sequestration, disruption of nucleocytoplasm transport and endoplasmic reticulum architecture, and mitochondrial dysfunction in different diseases. In addition, the expanded RNA can also give rise to noncanonical repeat- associated non-ATG (RAN) translation products. Thus, ridding cells of the mutant polyQ RNA/protein has become a major disease intervention and its success is thought to ameliorate any downstream pathomechanistic defects.

Significant progresses have been made through CRISPR-Cas genome editing and antisense oligonucleotides (ASOs) therapies in many polyQ diseases, especially in HD, SCA2 and SCA3. Efforts towards engineering high fidelity Cas proteins with various controller modules, split Cas proteins for efficient viral packaging, and non-viral delivery approaches are underway to improve precision and safety. ASOs have been successfully tested in HD, SCA1, SCA2, SCA3 and SCA7 cells or mouse models and with HD ASO entering Phase III clinical trial. The efficacy of ASO can vary from case to case and can largely depend on RNase H activity, accessibility of target sites, design chemistry and bioavailability in target tissue. Observations from clinical trials and animal studies indicate that targeting both mutant and normal alleles of the polyQ protein does not associate with any pathology ordeleterious side effects. In addition, no off-target effects were detected using CAG complementary ASOs in HD and SCA3 patient cells or HD Q175 mice. The aboveobservations have clearly outlined the possibility and benefit of using a single repeat-based agent to reduce mutant polyQ RNA and protein load across multiple diseases.

Since the 8-17 core cleaves the phosphodiester bond between ribonucleotides A and G, an 8-17 DNAzyme was designed that binds to and cleaves CAG repeat RNA. The designer DNAzyme cleaves its target in the presence of different ions and under physiological concentrations. The chemically stabilized DNAzyme retainsthe catalytic activity and knocks down or eliminates mutant polyQ RNA or protein load in cells from several polyQ disease origins and high molecular weight (HMW) ATXN3proteins in a SCA3 mouse model.

### Methods

General methods and DNA. Unmodified DNA oligonucleotides were purchased from Sigma Aldrich. PNA oligonucleotides were purchased from PNA Bio. LNA oligonucleotides were purchased from Qiagen. Q5 High fidelity DNA Polymerase (New England Biolabs) were used for routine PCR amplification. Restriction digestion and ligation were performed with FastDigest enzymes and T4 DNA Ligase Kit (Thermo Fisher Scientific) respectively. DNA plasmids were propagated in chemically competent Escherichia coli Stbl3 (Thermo Fisher Scientific) and sequenced at GENEWIZ.

HEK293 and SH-SY5Y cells were cultured at 37° C. with 5% CO2 in Dulbecco’s modified Eagle’s medium (DMEM, GIBCO) containing 10% non-inactivated fetal calf serum (FBS, GIBCO) and 100 U/ml penicillin-streptomycin (P/S, GIBCO). Normal (GM23973) and patient derived SCA1 (GM06927, 52/29 CAG repeats) and SCA3 (GM06151, 74/24 CAG repeats) fibroblast cells were purchased from Coriell Cell Repositories and cultured in DMEM supplemented with 10% FBS and 1% P/S. Fibroblasts used in this study are no more than 2 passages different across cell lines and are all below 15 passages for nucleofection. BMECs (brain microvascular endothelial cells, gift of Dr. Kyuson Yun) were cultured on plates coated with 2 µg/cm² fibronectin at 37° C. in Medium 199 (Gibco) containing 5% NuSerum, 5% FBS and 1% P/S. Normal WTC11 iPSCs (GM25256) were purchased from Coriell Cell Repositories. Different iPSC lines were cultured in TeSR™-E8™ (STEMCELL Technologies) on plates or round coverslips coated with Matrigel (Corning).

The expanded ATXN1 (gift of Dr. Huda Zoghbi), TBP (gift of Dr. Shihua Li), ATXN7 (gift of Dr. Harry Orr) and AR (Addgene #28235) genes were cloned from their parental backbones into pCMV-(DYKDDDDK)-N vector to acquire a FLAG tag (Clonetech). The pEGFP-Q74 (for HTTex1, #40262), pEGFP-C1-ATXN3-Q28 (#22122) and pEGFP-C1-ATXN3-Q84 (#22123) plasmids were purchased from Addgene.

Animals. All animal procedures were approved by the Houston Methodist Institutional Animal Care and Use Committee and conducted in compliance with the National Institutes of Health guidelines for the use of experimental animals. Genotyping was performed using tail biopsy DNA isolated prior to weaning. PCR was performed using Qiagen DNeasy Blood and Tissue Kit and primers recommended by the Jackson Laboratory to amplify a fragment of the ATXN3 transgene. Animals were matched for age, sex and weight for this study. Six-month old animals were injected with either saline or DNAzyme and brain tissues were harvested after one month for imaging and biochemical analyses. Before euthanization, blood was drawn and analyzed for clinical chemistry and hematology at the MD Anderson core facility. Different experimental stages — such as injection, brain harvest and biochemical assays — were carried out by different personnel to ensure data reproducibility.

In vivo Imaging System (I.V.I.S.) and stereotaxic injection. I.V.I.S. imaging was performed using a Caliper Xenogen 200 system and images were normalized using the Living Image Software.

DNAzyme was stereotaxically administered into the right lateral ventricle. Intracerebroventricular (i.c.v.) injections were performed on mice under intraperitoneal ketamine anesthesia. For each i.c.v. injection, a small incision was made to expose the Bregma and a small hole was drilled at the coordinates pre-determined by the NEUROSTAR software (mediolateral +1 mm; anterior-posterior -0.3 mm; dorsoventral -2.5 mm). Four hundred µg LNA8-17Dz9 was delivered at an infusion rate of 4 turns/min using a Hamilton syringe on a Global Biotech stereotaxic instrument. After the injection was complete, the needle was slowly retracted at a rate of 1 mm/min and animals were sutured and supplied with pain killer.

RNA in vitro transcription and cleavage assay. CAG_{X30} and CAGx42 was cloned into pcDNA3.1-hygro+ at NheI and HindIII sites, linearized at EcoRV site, and in vitro transcribed at 30° C. overnight using HiScribe T7 High Yield RNA Synthesis Kit (NEB) with approximately 500 ng of DNA input. The transcribed RNA was digested with TURBO DNaseI at 37° C. for 30 min before column purified using RNA Clean and Concentrator (Zymo Research).

RNA cleavage assays were performed in 1x Dz digestion buffer (50 mM Tris-HCl pH 7.5, 150 mM NaCl and varying concentrations of Mg²⁺ or Ca²⁺) with 100 nM target RNA and 500 nM 8-17 Dz at 37° C. for the indicated time. Samples were quenched in 2x TBE-Urea Loading Buffer (BioRad) mixed with 3x formamide dye (100% formamide, 0.5 M EDTA), boiled at 95° C. for 5 min, and loaded on 10% TBE-Urea gels. After running at 200 V for approximately 1 h, RNA cleavage was detected in SYBR Gold (Invitrogen, 1:50,000) on an Azure c400 Biosystem.

Transfection. HEK293 or SH-SY5Y cells were seeded at 700,000 per well (6-well format) approximately 16 h before transfection. Transfection was performed with 2 µg plasmid DNA ± 200 pmol 8-17Dz in the presence of 15 µl Lipofectamine 2000 (Invitrogen) according to the manufacturer’s instruction.

Fibroblasts were nucleofected using the P2 Primary Cell 4D-Nucleofector X Kit (100 µl format) on a 4D-Nucleofactor X unit (Lonza) according to the manufacturer’s instruction. One million cells combined ± 200 or 300 pmol DNAzyme were nucleofected under the program “CZ167” and plated in pre-warmed medium in each well. Analyses (immunoblot/IF/functional assays) were performed 48 h post transfection.

Each 100 pmol DNAzyme was transfected with 1.5 µl Lipofectamine Stem Reagent (Thermo Fisher Scientific) into iPSCs or differentiating iNeurons according to the manufacturer’s protocol.

RNA Isolation and quantitative RT-PCR (qRT-PCR). Total RNA was isolated using RNeazy Mini Kit (Qiagen), digested with TURBO DNaseI (Thermo Fisher Scientific), and purified using RNA Clean and Concentrator (Zymo Research) according to the manufacturer’s protocol. One microgram input RNA was reverse transcribed using iScript cDNA Synthesis Kit (Bio-Rad). PCR amplification was performed with 2.5 µl of diluted cDNA (1:3 dilution) on a CFX96 Real-time System (Bio-Rad). RNA expression was normalized against that of GAPDH and the relative normalized expression was calculated using CFX Manager 3.1 (Bio-Rad).

DCL64 liposome packaging. 1,2-Dipalmitoyl-sn-glycero-3-phosphocoline (DPPC) and cholesterol were purchased from Avanti Polar Lipids and poloxamer L64 was purchased from Sigma-Aldrich. DCL64 liposomes were prepared by mixing DPPC, cholesterol and Poly(ethylene glycose)-block-poly(propylene glycol)-block-poly(ethylene glycol) L64 (poloxamer L64) at a 5:3:7 weight ratio at room temperature. DNAzyme was mixed with DCL64 at a 1:10 weight ratio at room temperature. All DCL64 liposomes were prepared in Wheaton glass vials and mixed with excess tert-butanol at room temperature prior to freezing at -80° C. overnight. The DCL64 liposomes were then lyophilized overnight, using a FreeZone 2.5 lyophilizer (Labconco), until a thin film had formed and there was no evidence of liquid within the glass vial. DCL64 liposomes not needed for use immediately were stored at -20° C. The DCL64 liposomes were reconstituted with sterile 1X PBS to the desired concentration immediately before use.

DNA tetrahedron assembly and packaging. PAGE-purified DNA oligonucleotides (Table 1) were purchased from Sigma-Aldrich. DNA tetrahedra were generated by mixing equimolar quantities (1 µM) of each strand (± DNAzyme) in 1x TM buffer (20 mM Tris-HCl pH7.5, 50 mM MgCl₂-H2O), and the mixture was slowly cooled from 95° C. to 25° C. over 2 h. The assembled structure was characterized by 1% agarose gel and 4% native PAGE gel. Two assembly reactions were pooled (equivalent to 200 pmol of DNAzyme) for cell delivery.

TABLE 1

List of oligonucleotides used in this study. The catalytic core sequences are shown as bolded

| | | |
|---|---|---|
| 8-17Dz5 | GCTGTCCGAGCCGGACGAGCTGC | SEQ ID NO: 121 |
| 8-17Dz6 | TGCTGTCCGAGCCGGACGAGCTGCT | SEQ ID NO: 122 |
| 8-17Dz7 | CTGCTGTCCGAGCCGGACGAGCTGCTG | SEQ ID NO: 123 |
| 8-17Dz8 | GCTGCTGTCCGAGCCGGACGAGCTGCTGC | SEQ ID NO: 124 |
| 8-17Dz9 | TGCTGCTGTCCGAGCCGGACGAGCTGCTGCT | SEQ ID NO: 125 |
| 8-17Dz10 | CTGCTGCTGTCCGAGCCGGACGAGCTGCTGCTG | SEQ ID NO: 126 |
| 8-17Dz11 | GCTGCTGCTGTCCGAGCCGGACGAGCTGCTGCGC | SEQ ID NO: 127 |
| 8-17Dz12 | TGCTGCTGCTGTCCGAGCCGGACGAGCTGCTGCTGCT | SEQ ID NO: 128 |
| 8-17Dz13 | CTGCTGCTGCTGTCCGAGCCGGACGAGCTGCTGCTGCTG | SEQ ID NO: 129 |
| LNA8-17Dz9 | +T+G+C+T+GCTGTCCGAGCCGGACGAGCTG+C+T+G+C+T | SEQ ID NO: 130 |
| PNA8-17Dz9 | *T*G*C*T*G*C*T*G*T*C*C*G*A*G*C*C*G*G*A*C*G*A*G*C*T*G*C*T*G*C*T | SEQ ID NO: 131 |
| polyT20LNA8-7Dz9 | TTTTTTTTTTTTTTTTTTTT+T+G+C+T+GCTGTCCGAGCCGGACGAGCTG+C+T+G+C+T | SEQ ID NO: 132 |
| polyA20A20 | AAAAAAAAAAAAAAAAAAAAATTGCTGTATTGGCTCTGGTGATGCGTTAAAGGATCTCGTATAGCAGCTCAGTCCACTCGAAC | SEQ ID NO: 133 |
| B20 | CATAGTCAATAACGCATCACCAGAGCCAAATGACGACATCTGTGCGATGAAACCTAGCAGACC | SEQ ID NO: 134 |
| C20 | GAGATCCTATGACTATGGGTCTGCTAGGTACAGTCTGTCGCTTATGCACTAGAGCTGCTATAC | SEQ ID NO: 135 |
| D20 | TGTCGTCAAACAGCAATGTTCGAGTGGACAAGTGCATAAGCGACAGACTGATCATCGCACAGA | SEQ ID NO: 136 |
| qRT-PCR pimers for GAPDH | GACAGTCAGCCGCATCTTCT | SEQ ID NO: 137 |
| | GCGCCCAATACGACCAAATC | SEQ ID NO: 138 |
| qRT-PCR primers for GFP | CAAGATCCGCCACAACATCG | SEQ ID NO: 139 |
| | GACTGGGTGCTCAGGTAGTG | SEQ ID NO: 140 |

Protein isolation and immunoblot. Proteins were isolated from cells 48 h post transfection and lysed in Pierce IP Lysis Buffer (Thermo Fisher Scientific) supplemented with cOmplete Protease Inhibitor Cocktail (Roche) and PhosSTOP (Sigma-Aldrich) at 4° C. for 30 min. Soluble fractions were collected after centrifugation at 13,200 rpm at 4° C. for 15 min and quantified using Pierce BCA Protein Assay Kit (Thermo Fisher Scientific).

For immunoblot analysis, approximately 40 µg of proteins were denatured in 4x Laemmli Sample Buffer (Bio-Rad) and P-mercaptoethanol at 95° C. for 5 min before loaded on 4-15% SDS-PAGE gels (Bio-Rad). Proteins were transferred onto PVDF membranes (Bio-Rad) using a Mini Trans-Blot Electrophoretic Transfer Cell (Bio-Rad) at 100 V for approximately 1.5-2 h at 4° C. Membranes were incubated with blocking buffer (5% Blotting-Grade Blocker, Bio-Rad, in 1x TBST, 0.1% Tween 20) at room temperature for 1 h, followed by primary antibody incubation at 4° C. overnight and secondary antibody incubation at room temperature for 1 h. Chemilunimescent signals were detected using SuperSignal West Pico Chemiluminescent Substrate (Thermo Fisher Scientific) on an Azure c400 Biosystem and quantified using ImageJ.

Cortical tissues from mouse brain were flash frozen for protein analysis. Total protein lysate was extracted using mechanical homogenization in 500 µl RIPA buffer containing cOmplete mini protease inhibitor cocktail and PhoStop phosphatase inhibitor. Homogenate was incubated at 4° C. with agitation for 2 h prior to clearing the debris via centrifugation at 12,000 rpm for 20 min at 4° C. Supernatants were used for immunoblotting analysis.

Primary antibodies used in this study include mouse anti-ATXN1 (2F5, 1:1,000, Thermo Fisher Scientific), rabbit anti-ATXN2 (NBP1-90063, 1:200, Novusbio), mouse anti-ATXN3 (MAB5360, 1:1,000, Millipore), rabbit anti-ATXN3 (13505-1-AP, 1:500, Proteintech), mouse anti-FLAG (M2, 1:1,000, Sigma-Aldrich), mouse HRP-conjugated GAPDH (HRP-60004, Proteintech), mouse anti-GFP (GF28R, 1:1,000, Thermo Fisher Scientific), mouse anti-polyglutamine (MAB1574/5TF1-1C2, 1:1,000, Millipore), rabbit anti-Oct4 (#2750, 1:200, Cell Signaling Technology), rabbit anti-Sox2 (D6D9, 1:200, Cell Signaling Technology), rabbit anti-Nanog (D73G4, 1:200, Cell Signal Technnology), mouse anti-8-OHdG (E-8, 1:200, Santa Cruz Biotechnology), rabbit anti-ATXN7 (PA1-749, 1:1,000, Thermo Fisher Scientific), mouse anti-TBP (1TBP18, 1:1,000, Thermo Fisher Scientific), and mouse anti-p62/SQSTMl (D-3, 1:500, Santa Cruz Biotechnology).

iNeuron differentiation and harvest. iNeurons were differentiated as previously described. Briefly, WTC11 iPSCs with integrated hNGN2 were cultured on 6-well plates coated with Matrigel and induced in cortical neuron culture medium (DMEM/F 12 with HEPES, 1x B27 supplement, doxycycline 2 µg/ml). On day 3, cells were individualized by Accutase (STEMCELL Technologies), seeded on Matrigel coated 6-well plates, and transfected using Lipofectamine Stem Transfection Reagent with HTTex1-Q74 ± DNAzyme (total DNA: LipoStem ratio is 1: 2) according to the manufacturer’s protocol. Cells were harvested for immunoblot or imaging on day 5 and day 7.

Generation of SCA3 Ipsc. The study was approved by the University of Florida Institutional Review Board. The SCA3 subject was provided with the approved informed consent. Skin biopsy was performed by punch biopsy (6 mm in diameter) under local anesthesia. The skin specimens were placed in sterile DMEM medium supplemented with 20% FBS and 1% P/S at room temperature for transport to the lab. Biopsy specimens were processed into 0.5 mm cubes and placed into duplicate 25 cm2 flasks. The explants were allowed to air-dry for 30 min and 12 ml of primary culture medium (DMEM with 20% FBS) was added to the flask. The flasks were placed in a 37° C. 5% CO2 incubator. Medium was replenished after 7 days. When fibroblasts from adjacent explants started to merge, the flasks were treated with 0.05% Trypsin/EDTA and passed to a 75 cm2 flask. These cells were designated as passage 1. Passage 3 fibroblasts were used for reprogramming.

Reprogramming was performed by a non-integrating method using CytoTune Sendai Reprogramming Kit (Thermo Fisher Scientific) on the fibroblasts according to the manufacturer’s protocol as previously published. Isolated iPSC clones were cultured on either vitronectin- or Matrigel-coated plates in TeSR-E8 medium (STEMCELL Technologies).

Fluorescent microscopy and immunofluorescence (IF). To evaluate DNAzyme’s cellular uptake, BMEC or SH-SY5Y cell were incubated with naked or DCL64-packaged Cy3-LNA8-17Dz9 for 48 h, washed five times with PBS (5 min each), fixed in 4% Paraformaldehyde (PFA) Solution in PBS (Thermo Fisher Scientific) for 10 min, permeabilized in 0.2% Triton X-100 in PBS for 10 min, and mounted in Mounting Medium with DAPI for Fluorescence (Vectorshield). Images were taken on a Nikon A1 Confocal Imaging system.

IF was carried out 48 h post transfection. Fibroblasts, iPSCs or iNeurons on coverslips were washed with PBS, fixed in 4% PFA, permeabilized in 0.2% Triton X-100 in PBS, and blocked in 5% BSA in PBST (0.05% Tweeen 20). Primary antibody incubation was carried out at 4° C. overnight, followed by fluorophore-tagged secondary antibody incubation at room temperature for 1 h before mounting in DAPI medium. A Nikon A1 Confocal Imaging system was used.

After brain harvest, sagittal sections of all mouse brains were fixed in 4% PFA for 48 h, cryoprotected in a sucrose gradient and embedded in OCT. Cryostat sections from OCT embedded tissue were cut at 10 µm thickness and mounted on slides. Sections were fixed in 4% PFA, washed in PBS and blocked in 5% normal goat serum, 0.2% Triton X-100 in PBS. Sections were then incubated in mouse anti-GFAP antibody (1:500, sc-33673, Santa Cruz Biotechnology) at 4° C. overnight. After washing, the sections were incubated in secondary antibody, washed in PBS and mounted in Mounting Medium with DAPI (Vectorshield). Slides were imaged with a Nikon A1 Confocal Imaging system.

Cell proliferation assay. Cell proliferation was assessed using the Aqueous One Solution Cell Proliferation Assay (Promega). In brief, 5,000 cells (± nucleofected DNAzyme) were plated in each 96 well and treated with 20 µl MTS at 37° C. for 3 h. Absorbance at 490 nm was recorded on a PerkinElmer EnSpire Mulitmode Plate Reader.

Mitochondrial membrane potential assay. Mitochondrial membrane potential was measured using the JC-10 dye according to the manufacturer’s protocol (G-Biosciences). In brief, 5,000 cells (from the same nucleofection used for cell proliferation assays) were plated in each 96 well, incubated with 100 µl JC-10 solution (1: 200 dilution) for 20 min at 37° C., washed twice with 1x MMP solution (1: 5 dilution), and recorded at excitation/emission 535 nm /595 nm and 485 nm/535 nm on the EnSpire plate reader. The red-to-green fluorescence ratio was used for plotting the graph.

Statistical Analysis and image processing. Statistical analyses of all data were performed using t test in GraphPad Prism Version 7.03 (*P < 0.05, **P < 0.01, ***P < 0.001).

Quantification of p62 aggresome/cell was performed in Fuji ImageJ. p62 foci were counted using “Find maxima” with the threshold set to 60. Cells were counted in 8-bit images with threshold set to 30-255 and particle size set to 60-infinity.

### Results

### Designer DNAzyme 8-17 (8-17 Dz) Cleaves CAG Repeat RNA Biochemically

Three 8-17 Dzs with varying lengths of CAG complementing arms were initially designed and tested for cleavage efficiency of in vitro transcribed CAGx30 RNA (FIG. 1A). Although the highest activity of 8-17 Dz core is normally observed in the presence of Pb²⁺, Mg²⁺ was initially screened - one of the most abundant metal ions in biological fluids. Amongst the three 8-17 Dz designs, the 8-17 Dz with 9 nt binding arms (8-17 Dz9) exhibits the highest RNA cleavage activity across the tested range of Mg²⁺ concentrations over 1 h (FIGS. 1B and 8). Additional alterations of RNA binding arm’s length changed the catalytic property of 8-17 Dz to different extents (FIG. 9). Further analysis using Ca²⁺ as a cofactor shows superior RNA cleavage efficiency compared to Mg²⁺ (FIGS. 1B and 8). To test if the 8-17 Dz9 can work under physiologically relevant Mg²⁺ concentrations, RNA cleavage was assayed in the presence of 5 mM Mg²⁺ (FIG. 10). Approximately 50% of the target CAGx30 RNA were cleaved by 8-17 Dz9 in 5 h. The same 8-17 catalytic core has been successfully used to detect physiological Ca²⁺/Mg²⁺ levels in undiluted human blood serum. Taken together, the repeat-based 8-17 Dz9 efficiently cleaves CAG RNA biochemically under low ionic strength and is activated by both Ca²⁺ and Mg²⁺ ions, generating a compounding effect on CAG RNA cleavage in cells.

### Locked Nucleic Acid Modification Retains 8-17 Dz’s Catalytic Activity

Unmodified DNAzyme can be degraded by cellular nucleases; increased stability and affinity towards its target RNA can offer persistent therapeutic benefit in cells. Amongst the repertoire of chemical modifications, the locked nucleic acid (LNA) and peptide nucleic acid (PNA) were chosen for base substitutions in 8-17 Dz9 due to their strong binding affinity and incompatibility with RNase H. The terminal five nucleotides of each binding arm were substituted to LNA to create the LNA8-17Dz9 and all nucleic acids were substituted to PNA to create the PNA8-17Dz9 (FIG. 1A). To test if these modifications can affect DNAzyme function, RNA cleavage assays were performed at both high and low Mg²⁺ concentrations. The LNA8-17Dz9 retains comparable catalytic activity to that of the unmodified 8-17 Dz9 (FIGS. 1B and 10). The PNA8-17Dz9 showed reduced RNA cleavage compared to the unmodified 8-17Dz9 (FIGS. 1B and 10), due to unfavorable folding of the peptidyl catalytic core, as global folding efficiency of DNAzyme has been linked to catalytic activity. The DNAzyme’s cleavage efficiency on two different in vitro transcribed CAG repeat lengths, CAGx30 and CAGx42 was further compared (FIG. 11). The LNA8-17Dz9 biochemically cleaves both RNA repeats with similar catalytic profiles.

### LNA8-17Dz9 Significantly Knocks Down or Eliminates Multiple Mutant polyQ Proteins in HEK293 Cells

Huntington’s disease is the most common inherited neurodegenerative disease and is caused by CAG repeat expansion in the first exon of Huntingtin (HTTex1). The mutant HTT (mutHTT) undergoes extensive autolysis and produces exon 1 fragments that aggregates in neuronal nuclei of brain tissue. To test if DNAzyme can knock down mutHTTex1, an HTTex1 fragment containing 74 Qs (HTTex1- Q74) was co-transfected with the three 8-17Dz9 designs in HEK293 cells for 48 h. The highest HTTex1-Q74 RNA knockdown was observed by LNA8-17Dz9, followed by unmodified 8-17 Dz9 and PNA8-17Dz9 (FIG. 12). At a protein level, the LNA8-17Dz9 showed the highest knockdown, followed by PNA8-17Dz9 and unmodified 8-17Dz9 (FIG. 2A). The observed protein knockdown is not due to competition of DNAzyme with polyQ plasmids during co-transfection by using a control DNA fragment of similar length (FIG. 13). The observed large variability in PNA8-17Dz9 transfected cells can be due to inconsistent packaging between the neutral amide PNA backbone and the cationic transfection lipids. Given that the LNA8-17Dz9 is most effective in reducing RNA and protein load of mutHTTex1, it was used for the subsequent experiments.

CAG expansion in ataxin 1 (ATXN1), TATA binding protein (TBP), ataxin 7 (ATXN7) and androgen receptor (AR) causes SCA1, SCA17, SCA7 and SBMA respectively. Each mutant polyQ protein was cloned to acquire a FLAG tag and subsequently co-transfected with LNA8-17Dz9 in HEK293 cells for immunoblot analysis. The LNA8-17Dz9 completely eliminates TBP-Q94 and ATXN7-Q61 protein expression and significantly reduces ATXN1-Q83 and AR-Q40 protein levels by approximately 80% (FIG. 2B). The calculated dosage of DNAzyme and cell density indicates that between 8-12 million DNAzyme molecules are available for intracellular targeting, if a 50% transfection is achieved. To evaluate allele specificity by LNA8-17Dz9, the DNAzyme was co-transfected with either ATXN3-Q28 or ATXN3-Q84 (mutant ATXN3 causes SCA3) in HEK293 cells. The LNA8-17Dz9 completely eliminates both proteins (FIG. 14). In the subsequent competitive cleavage experiment, an equal weight of ATXN3-Q28 and ATXN3-Q84 plasmids was co-transfected with LNA8-17Dz9 for immunoblotting. When both proteins are present in HEK293 cells, preferential targeting of ATXN3-Q84 over Q28 was not observed (FIG. 14). Taken together, the LNA8-17Dz9 efficiently clears a panel of mutant polyQ proteins in HEK293 cells.

### LNA8-17Dz9 Significantly Reduces polyQ Protein Load in Neuroblastoma Cells and iNeurons

To test if LNA8-17Dz9 can work in neuron-like cells, HTTex1-Q74 and LNA8-17Dz9 were co-transfected into SH-SY5Y neuroblastoma cells. A >90% target protein knockdown was observed (FIG. 3A). Striatal and cortical neurons exhibit prominent cell loss in postmortem HD brain tissues. To test efficacy in neurons, a wildtype (wt) human iPSC line stably expressing a doxycycline-inducible human neurogenin 2 (WTC1 1-hNGN2) at the safe-harbor AAVS1 locus was utilized. Upon induction, the WTC11- hNGN2 iPSCs rapidly differentiate into cortical excitatory neurons (iNeurons). On a 5-day differentiation scheme, the iNeurons produced dendrites and expressed neuronal markers NeuN and MAP2 (FIG. 3B). Co-transfection of HTTex1-Q74 and 200 pmol LNA8-17Dz9 on day 3 reduced the target protein load by 90% (FIG. 3C). On a 7-day differentiation scheme (transfection on day 3 and harvest on day 7), the iNeurons lost the transgene expression entirely due to the nature of transient transfection (FIG. 15). Taken together, the LNA8-17Dz9 reduces approximately 90% of the polyQ protein load in neuroblastoma cells and iPSC-derived cortical neurons.

### LNA8-17Dz9 Targets Both Normal and Expanded ATXN3 Alleles Without Affecting p62-Aggresome Levels in SCA3 iPSCs

SCA3 is the most common inherited ataxia and is caused by a CAG repeat expansion in the ATXN3 gene. Although its role as a deubiquitinase has been associated with aggresome formation, autophagy, antiviral response, and transcription-coupled DNA repair, different Ataxin3-knockout (KO) mice exhibit normal viability and fertility, indicating that its function is c essential. Thus, lowering the expression of both wildtype and mutant ATXN3 can be well tolerated. A patient- derived SCA3 iPSC line that expresses pluripotency markers Oct4, Sox2 and Nanog and has a repeat size close to CAGX80 were generated (FIG. 3D and FIG. 16). Transfection of 200 pmol or 300 pmol of LNA8-17Dz9 showed similar knockdown of both wtATXN3 and mutATXN3 proteins by approximately 40% (FIG. 3E); this non-allele-specific knockdown in iPSCs is consistent with observations in the overexpression system (FIG. 14). The autophagy-linked ubiquitin-binding shuttle protein p62 (SQSTM1) is known to co-localize with both wt and mutATXN3 proteins and promote perinuclear aggresome formation. The non-allele-specific knockdown of ATXN3 in iPSCs did not show significant differences in p62-aggresome formation from untreated SCA3 iPSCs or normal WTC11 iPSCs (FIG. 17). Taken together, the LNA8-17Dz9 effectively lowers both wildtype and mutant ATXN3 to a similar extent in SCA3 iPSCs without affecting aggresome formation and by inference p62-dependent autophagy.

### Allele Specific Targeting of Mutant polyQ Protein can be Achieved in Patient-Derived Fibroblasts

To further investigate if LNA8-17Dz9 can achieve allele specificity, SCA1 and SCA3 patient-derived fibroblasts were nucleofected with either 200 or 300 pmol LNA8-17Dz9 for 48 h. In both cell lines, significant target knockdown (ATXN1 RNA in SCA1 and ATXN3 RNA in SCA3) were observed (FIG. 18). In SCA1 fibroblasts, the LNA8-17Dz9 preferentially reduces the mutATXNl protein load by 40-60% without affecting the wtATXN1level (FIG. 4). It did not alter the wtATXN3 level but reduced the wtATXN2 level by approximately 30-40%. Similarly, the LNA8-17Dz9 treatment did not alter the wtATXN7 level but reduced wtTBP to a level comparable to that in normal fibroblasts (FIG. 4). Even though ATXN3, ATXN2, TBP and ATXN7 all contain short CAG repeats, DNAzyme can affect their protein levels differentially (FIG. 4). Given that mutATXNl can undergo aggregation, inclusion fraction of protein post DNAzyme treatment was assay for and mutATXNl knockdown by DNAzyme as with the insoluble fraction was observed (FIG. 19). In SCA3 fibroblasts, the LNA8-17Dz9 knocks down both wtATXN3 and mutATXN3 by 40-50%, closely tracking the results obtained in SCA3 iPSCs (FIG. 5 vs. FIG. 3E). Consistent with SCA1 cells, the wtATXN1and wtATXN7 levels were not affected by DNAzyme treatment while the wtATXN2 level was reduced by approximately 40% and the wtTBP to a similar level as observed in normal fibroblasts (FIG. 5). The low expression level of wtATXN7 in both fibroblast cell lines coincides with that observed in HEK293 cells (FIG. 2B). The reason as to why DNAzyme suppressed wtATXN3 in SCA3 fibroblasts but not in SCA1 fibroblasts can be due to differences in wt allelic copies, as SCA3 fibroblasts have only one wtATXN3 allele while the tested SCA1 fibroblasts have two. Such dosing effect can be sensitive to DNAzyme treatment. In addition, the observed knockdown of both allele and non-allele selective manners requires further mechanistic investigations on the targeting moiety.

The DNAzyme-treated SCA3 fibroblasts were subject to further cell proliferation studies, given that potential off-target knockdown of wtATXN3, wtATXN2 and wtTBP was observed. The LNA8-17Dz9 treatment consistently improved SCA3 cell survival (even better survival than wt cells at low concentration, FIG. 6A). However, the reduced rescue of cell survival at a higher DNAzyme dosage (300 pmol) can indicate toxicity. The above data show that the on-target reduction of mutant polyQ protein benefits SCA3 cells and clearly outweighs the limited off-target cleavage. PINK1, PARKIN and ATXN3 play important roles in mitochondrial maintenance and clearance. PINK1 is specifically activated by mitochondrial membrane potential depolarization, and recruits PARKIN to and activates PARKIN’s ubiquitinase activity on damaged mitochondria. ATXN3 deubiquitinates PARKIN directly and reduces the extent of PARKIN ubiquitination in cells. To see if knockdown of wtATXN3 in SCA3 fibroblasts by DNAzyme can off-set PARKIN’s ubiquitination on depolarized mitochondria and lead to accumulation of low-quality mitochondria, mitochondrial membrane potential in DNAzyme-treated SCA3 fibroblasts was measured and no change was observed with respect to untreated or normal cells (FIG. 6B). As a result, no oxidative DNA damage from mitochondrion-derived reactive oxygen species was detected using 8-hydroxy-2-deoxyguanosine antibodies (8-OHdG, FIG. 20). Taken together, allele specific targeting of mutant polyQ protein can be achieved and limited off-target cleavage does not compromise cell survival or mitochondrial function in patient-derived fibroblasts.

Enhanced delivery to target tissues/cells is an important area of research for translational application of DNAzyme. Two packaging/delivery methods were explored in this study, namely the liposome DCL64 formulation and DNA tetrahedron. Despite the significantly increased cellular uptake in both blood brain barrier endothelial and neuroblastoma cells (FIG. 21), the DCL64-packaged DNAzyme only reduced the mutant polyQ protein load by 20% (FIG. 21F). Given that the fluorescently tagged DNAzyme is retained within cells (FIGS. 21A-21D), the payload is not sufficiently released from the endosome, possibly due to a lack of ionizable cations in the formulation. Various DNA tetrahedral nanoparticles have successfully delivered payload into glioma, kidney or alveolar epithelial cells. However, the biological effect after delivery remains largely uninvestigated. DNA tetrahedron-packaged DNAzyme did not significantly knock down target protein in neuron-like cells, indicating that unfacilitated endocytosis of DNA nanoparticle is insufficient to achieve cellular efficacy(FIG. 21F). With recent advances in lipid nanoparticle formulation and cell penetrating peptide conjugation, it is possible to broaden DNAzyme’s biodistribution and bioavailability in animal models, even though a single or repeated injection of naked oligonucleotides already leads to a wide CNS spread in mice.

Stereotaxically injected LNA8-17Dz9 knocks down high molecular weight ATXN3 in vivo.

To evaluate the stability of LNA8-17Dz9 in vivo, a low dosage (25 µg) of Cy5-labeled DNAzyme was administered in the right ventricle of 6-week old SCA3 MJD84.2 mice by stereotaxic injection. Amongst several well established SCA3 mouse models, the transgenic MJD84.2 mouse was chosen for the following studies because the YAC construct harbors a human ATXN3 gene (with Q3KQ80 repeats) under its native promoter and the mouse exhibits disease-relevant neuropathological phenotype and behavioral abnormalities from 4 weeks of age. I.V.I.S. imaging was performed 48 h to 42 days post injection and indicates that the low-dose LNA8-17Dz9 is detectable in mouse brain for at least 1 month (FIG. 7A). Six-week old SCA3 mice (n=4) were subsequently injected with an intermediate dose (400 µg) of LNA8-17Dz9 in the same brain region. One month post injection, three of the four injected mice were harvested for immunoblotting and one for IF imaging. For comparison, age-matched WT mice and SCA3 mice injected with saline (n=4 for each) were used as controls. All the DNAzyme-injected mice survived and showed no signs of toxicity or apparent gliosis (FIG. 23). Immunoblotting shows that the LNA8-17Dz9 significantly reduced the HMW ATXN3 accumulation by approximately 50% (FIG. 7B). At the tested dosage, changes were not observed in the level of soluble, monomeric mutATXN3 and wtATXN3 but a 20% reduction of wtATXN2 (FIG. 7B). The wtTBP level post DNAzyme treatment was not significantly different from that of WT mouse (FIG. 7B), consistent with the observations in patient-derived fibroblast cells (FIGS. 4 and 5).

### Example 2 Designer DNAzymes Directly Eliminate Essential Viral RNA in Coronaviridae

The 2019 coronavirus (COVID-19 or SARS-CoV-2) originated in Wuhan quickly spread beyond China. Since the pathogenesis of this virus is not well understood, there are limited front-line diagnostic tools or therapeutic agents available to fight this pandemic. The estimated timeline for an effective treatment for SARS-CoV-2 is at least one year away, despite tremendous global resources are dedicated to drug development. Antibody- or antagonistic peptide-based vaccines may benefit patients by neutralizing circulating viruses at protein epitopes, however, they may not stop viral replication inside cells due to their limited cell-penetrating capacity. In addition, evidence indicates that the virus is actively acquiring new mutations, which can help it escape antiviral molecules and render existing vaccines inefficacious. These therapeutic limitations also apply to other deadly RNA viruses such as SARS-CoV (Severe Acute Respiratory Syndrome-related coronavirus) or MERS-CoV (Middle East Respiratory Syndrome).

An RNA-cleaving DNAzyme that can eliminate repeat CAG RNA from several polyglutamine diseases in vitro and in cellula was developed. The DNAzyme has a catalytic core and two RNA binding arms that can be reprogrammed to anneal to different RNA targets (in this case different SARS-CoV-2 viral RNA). It cleaves the target RNA under physiological ionic strength without requiring any protein component. The present study shows that the DNAzyme can be used to target the RNA genome of SARS-CoV-2, especially the highly conserved regions within five essential genes, namely the papain-like protease (PLpro), 3-chymotrypsin-like protease (3CLpro), RNA-dependent RNA polymerase (RdRp), helicase and spike (S); without these genes the virus cannot replicate or survive in human cells. It can be particularly important to target the RdRp gene because this protein is responsible for transcribing other viral RNA into mRNA for further translation in the host cell. The advantages of our DNAzyme approach are: (1) the DNAzyme targets highly conserved RNA regions that are unlikely to mutate (even under evolutionary pressure), (2) these RNA regions are conserved across the coronaviridae family and can be used to treat different viral outbreaks (e.g., SARS-CoV and SARS-CoV-2), and (3) the DNAzyme works within cells before viral proteins are made, thus terminating the viral life cycle before spreading. The approach can be used for treating SARS-CoV-2 because other DNAzymes with different catalytic cores have been used to inhibit the replication of RNA viruses. For instance, the DNAzyme that cleaves between G and U inhibits the Japanese encephalitis viral replication in mouse brain and hepatitis C gene expression in human cells. The DNAzyme that cleaves between U and G inhibits hepatitis B and influenza viral gene expression in human cells by targeting the translation start codon. Given that the AG site targeted by our DNAzyme is more prevalent in the viral genome (AG is present in the stop codon (UAG), glutamine (CAG), lysine (AAG) and glutamate (GAG)) than the start codon (AUG), the DNAZyme has a broader targetability for RNA viruses.

The example analyzed the viral genome of 4 SARS-CoV (AY508724, AY485277, AY390556 and AY278489), 19 SARS-CoV-2 (including 5 strains in the US: MN988713.1 -Illinois, MN985325.1 - Washington, MN997409.1 - Arizona, MN994468.1 - California and MN994467.1 - California; 13 strains in China: MN975262.1, LR757995.1, MN938384.1, MN996527.1, LR757996.1, MN988669.1, MN996528.1, MN908947.3, MN988668.1, MN996530.1, MN996531.1, MN996529.1 and LR757998.1; 1 strain in Australia: MT007544.1), and 1 MERS-CoV (JX869059). DNA (RNA) sequences encoding PLpro (SEQ ID NOs: 1-24), 3CLpro (SEQ ID NOs:25-48), RdRp (SEQ ID NOs:49-72), helicase (SEQ ID NOs:73-96) and S (SEQ ID NOs:97-120) proteins were aligned using Multalin. Different SARS-CoV and SARS-CoV-2 strains show highly conserved regions in these genes, while MERS-CoV shows a low level of similarity to SARS-CoV and SARS-CoV-2. The target selection criteria include the following parameters: (1) a central AG site (adenine-guanine) for DNAzyme cleavage, (2) the AG site must be flanked by at least 9 absolutely conserved nucleotides on both sides, and (3) the DNAzyme must target both SARS-CoV and SARS-CoV-2 viral RNA. The target sites and their DNAzyme designs are listed in FIG. 23.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

SEQ ID NOs: 1-24 PLpro DNA/RNA from 4 SARS-CoV strains, 19 SARS-CoV-2 and 1 MERS-CoV strain.

SEQ ID NOs: 25-48 3CLpro DNA/RNA from 4 SARS-CoV strains, 19 SARS-CoV-2 and 1 MERS-CoV strain.

SEQ ID NOs: 49-72 RdRp DNA/RNA from 4 SARS-CoV strains, 19 SARS-CoV-2 and 1 MERS-CoV strain

SEQ ID NOs: 73-96 helicase DNA/RNA from 4 SARS-CoV strains, 19 SARS-CoV-2 and 1 MERS-CoV strain.

SEQ ID NOs: 97-120 S DNA/RNA from 4 SARS-CoV strains, 19 SARS-CoV-2 and 1 MERS-CoV strain.

SEQ ID NO: 121 8-17Dz5

GCTGTCCGAGCCGGACGAGCTGC

SEQ ID NO: 122 8-17Dz6

TGCTGTCCGAGCCGGACGAGCTGCT

SEQ ID NO: 123 8-17Dz7

CTGCTGTCCGAGCCGGACGAGCTGCTG

SEQ ID NO: 124 8-17Dz8

GCTGCTGTCCGAGCCGGACGAGCTGCTGC

SEQ ID NO: 125 8-17Dz9

TGCTGCTGTCCGAGCCGGACGAGCTGCTGCT

SEQ ID NO: 126 8-17Dz10

CTGCTGCTGTCCGAGCCGGACGAGCTGCTGCTG

SEQ ID NO: 127 8-17Dz11

GCTGCTGCTGTCCGAGCCGGACGAGCTGCTGCTGC

SEQ ID NO: 128 8-17Dz12

TGCTGCTGCTGTCCGAGCCGGACGAGCTGCTGCTGCT

SEQ ID NO: 129 8-17Dz13

CTGCTGCTGCTGTCCGAGCCGGACGAGCTGCTGCTGCTG

SEQ ID NO: 130 LNA8-17Dz9

+T+G+C+T+GCTGTCCGAGCCGGACGAGCTG+C+T+G+C+T

SEQ ID NO: 131 PNA8-17Dz9

*T*G*C*T*G*C*T*G*T*C*C*G*A*G*C*C*G*G*A*C*G*A*G*C*T
*G*C*T*G*C*T

SEQ ID NO: 132 polyT20LNA8-17Dz9

TTTTTTTTTTTTTTTTTTTT+T+G+C+T+GCTGTCCGAGCCGGACGAGCT
G+C+T+G+C+T

SEQ ID NO: 133 polyA20A20

AAAAAAAAAAAAAAAAAAAAATTGCTGTATTGGCTCTGGTGATGCGTTAA
AGGATCTCGTATAGCAGCTCAGTCCACTCGAAC

SEQ ID NO: 134 B20

CATAGTCAATAACGCATCACCAGAGCCAAATGACGACATCTGTGCGATGA
AACCTAGCAGACC

SEQ ID NO: 135 C20

GAGATCCTATGACTATGGGTCTGCTAGGTACAGTCTGTCGCTTATGCACT
AGAGCTGCTATAC

SEQ ID NO: 136 D20

TGTCGTCAAACAGCAATGTTCGAGTGGACAAGTGCATAAGCGACAGACTG
ATCATCGCACAGA

SEQ ID NO: 137 qRT-PCR pimers for GAPDH

GACAGTCAGCCGCATCTTCT

SEQ ID NO: 138 qRT-PCR pimers for GAPDH

GCGCCCAATACGACCAAATC

SEQ ID NO: 139 qRT-PCr primers for GFP

CAAGATCCGCCACAACATCG

SEQ ID NO: 140 qRT-PCr primers for GFP

GACTGGGTGCTCAGGTAGTG

SEQ ID NO: 141

TCATGTGGTAGTGTTGGTT

SEQ ID NO: 142

GATGTTGTTAGACAATGCT

SEQ ID NO: 143

TGCGGTGTAAGTGCAGCCC

SEQ ID NO: 144

GTGCGGCACAGGCACTAGT

SEQ ID NO: 145

ACAGGCACTAGTACTGATG

SEQ ID NO: 146

TACATAATCAGGATGTAAA

SEQ ID NO: 147

GTTTCTTTAAGGAAGGAAG

SEQ ID NO: 148

CTTTAAGGAAGGAAGTTCT

SEQ ID NO: 149

AAGGAAGGAAGTTCTGTTG

SEQ ID NO: 150

AATGGGGTAAGGCTAGACT

SEQ ID NO: 151

GGTAAGGCTAGACTTTATT

SEQ ID NO: 152

TGAGTTATGAGGATCAAGA

SEQ ID NO: 153

TGAGGATCAAGATGCACTT

SEQ ID NO: 154

TGAATCTTAAGTATGCCAT

SEQ ID NO: 155

TATGCCATTAGTGCAAAGA

SEQ ID NO: 156

GCAAAGAATAGAGCTCGCA

SEQ ID NO: 157

AAAGAATAGAGCTCGCACC

SEQ ID NO: 158

TCGCACCGTAGCTGGTGTC

SEQ ID NO: 159

TCTATCTGTAGTACTATGA

SEQ ID NO: 160

GCCGCCACTAGAGGAGCTA

SEQ ID NO: 161

CGCCACTAGAGGAGCTACT

SEQ ID NO: 162

GTTGGACTGAGACTGACCT

SEQ ID NO: 163

ATCCTAATCAGGAGTATGC

SEQ ID NO: 164

CTAATCAGGAGTATGCTGA

SEQ ID NO: 165

GGGAACCTGAGTTTTATGA

SEQ ID NO: 166

AGTTTTATGAGGCTATGTA

SEQ ID NO: 167

CTTTGAAAAAGGTGACTAT

SEQ ID NO: 168

TCTCAGATGAGTTTTCTAG

SEQ ID NO: 169

GAGTTTTCTAGCAATGTTG

SEQ ID NO: 170

GTACTGGTAAGAGTCATTT

SEQ ID NO: 171

TTCTCTATGAGAACCAAAA

SEQ ID NO: 172

AACCAACATCCGAGCCGGACGAACCACATGA

SEQ ID NO: 173

AGCATTGTTCCGAGCCGGACGAAACAACATC

SEQ ID NO: 174

GGGCTGCATCCGAGCCGGACGATACACCGCA

SEQ ID NO: 175

ACTAGTGCTCCGAGCCGGACGAGTGCCGCAC

SEQ ID NO: 176

CATCAGTATCCGAGCCGGACGAAGTGCCTGT

SEQ ID NO: 177

TTTACATCTCCGAGCCGGACGAGATTATGTA

SEQ ID NO: 178

CTTCCTTCTCCGAGCCGGACGATAAAGAAAC

SEQ ID NO: 179

AGAACTTCTCCGAGCCGGACGATCCTTAAAG

SEQ ID NO: 180

CAACAGAATCCGAGCCGGACGATCCTTCCTT

SEQ ID NO: 181

AGTCTAGCTCCGAGCCGGACGATACCCCATT

SEQ ID NO: 182

AATAAAGTTCCGAGCCGGACGAAGCCTTACC

SEQ ID NO: 183

TCTTGATCTCCGAGCCGGACGACATAACTCA

SEQ ID NO: 184

AAGTGCATTCCGAGCCGGACGATGATCCTCA

SEQ ID NO: 185

ATGGCATATCCGAGCCGGACGATAAGATTCA

SEQ ID NO: 186

TCTTTGCATCCGAGCCGGACGAAATGGCATA

SEQ ID NO: 187

TGCGAGCTTCCGAGCCGGACGAATTCTTTGC

SEQ ID NO: 188

GGTGCGAGTCCGAGCCGGACGACTATTCTTT

SEQ ID NO: 189

GACACCAGTCCGAGCCGGACGAACGGTGCGA

SEQ ID NO: 190

TCATAGTATCCGAGCCGGACGAACAGATAGA

SEQ ID NO: 191

TAGCTCCTTCCGAGCCGGACGAAGTGGCGGC

SEQ ID NO: 192

AGTAGCTCTCCGAGCCGGACGACTAGTGGCG

SEQ ID NO: 193

AGGTCAGTTCCGAGCCGGACGACAGTCCAAC

SEQ ID NO: 194

GCATACTCTCCGAGCCGGACGAGATTAGGAT

SEQ ID NO: 195

TCAGCATATCCGAGCCGGACGACCTGATTAG

SEQ ID NO: 196

TCATAAAATCCGAGCCGGACGACAGGTTCCC

SEQ ID NO: 197

TACATAGCTCCGAGCCGGACGACATAAAACT

SEQ ID NO: 198

ATAGTCACTCCGAGCCGGACGATTTTCAAAG

SEQ ID NO: 199

CTAGAAAATCCGAGCCGGACGACATCTGAGA

SEQ ID NO: 200

CAACATTGTCCGAGCCGGACGAAGAAAACTC

SEQ ID NO: 201

AAATGACTTCCGAGCCGGACGATACCAGTAC

SEQ ID NO: 202

TTTTGGTTTCCGAGCCGGACGACATAGAGAA

SEQ ID NO: 203, 3CLpro (147/306), left arm,

AACCAACA

SEQ ID NO: 204, 3CLpro (298/306), left arm,

AGCATTGT

SEQ ID NO: 205, RdRp (15/932), left arm,

GGGCTGCA

SEQ ID NO: 206, RdRp (24/932), left arm,

ACTAGTGC

SEQ ID NO: 207, RdRp (27/932), left arm,

CATCAGTA

SEQ ID NO: 208, RdRp (357/932), left arm,

TTTACATC

SEQ ID NO: 209, RdRp (430/932), left arm,

CTTCCTTC

SEQ ID NO: 210, RdRp (432/932), left arm,

AGAACTTC

SEQ ID NO: 211, RdRp (433/932), left arm,

CAACAGAA

SEQ ID NO: 212, RdRp (511/932), left arm,

AGTCTAGC

SEQ ID NO: 213, RdRp (513/932), left arm,

AATAAAGT

SEQ ID NO: 214, RdRp (522/932), left arm,

TCTTGATC

SEQ ID NO: 215, RdRp (524/932), left arm,

AAGTGCAT

SEQ ID NO: 216, RdRp (545/932), left arm,

ATGGCATA

SEQ ID NO: 217, RdRp (549/932), left arm,

TCTTTGCA

SEQ ID NO: 218, RdRp (553/932), left arm,

TGCGAGCT

SEQ ID NO: 219, RdRp (553/932), left arm,

GGTGCGAG

SEQ ID NO: 220, RdRp (557/932), left arm,

GACACCAG

SEQ ID NO: 221, RdRp (563/932), left arm,

TCATAGTA

SEQ ID NO: 222, RdRp (583/932), left arm,

TAGCTCCT

SEQ ID NO: 223, RdRp (584/932), left arm,

AGTAGCTC

SEQ ID NO: 224, RdRp (801/932), left arm,

AGGTCAGT

SEQ ID NO: 225, RdRp (875/932), left arm,

GCATACTC

SEQ ID NO: 226, RdRp (876/932), left arm,

TCAGCATA

SEQ ID NO: 227, RdRp (919/932), left arm,

TCATAAAA

SEQ ID NO: 228, RdRp (922/932), left arm,

TACATAGC

SEQ ID NO: 229, Helicase (202/601), left arm,

ATAGTCAC

SEQ ID NO: 230, Helicase (261/601), left arm,

CTAGAAAA

SEQ ID NO: 231, Helicase (264/601), left arm,

CAACATTG

SEQ ID NO: 232, Helicase (288/601), left arm,

AAATGACT

SEQ ID NO: 233, S (918/1274), left arm,

TTTTGGTT

SEQ ID NO: 234, 3CLpro (147/306), right arm,

ACCACATGA

SEQ ID NO: 235, 3CLpro (298/306), right arm,

AACAACATC

SEQ ID NO: 236, RdRp (15/932), right arm,

TACACCGCA

SEQ ID NO: 237, RdRp (24/932), right arm,

GTGCCGCAC

SEQ ID NO: 238, RdRp (27/932), right arm,

AGTGCCTGT

SEQ ID NO: 239, RdRp (357/932), right arm,

GATTATGTA

SEQ ID NO: 240, RdRp (430/932), right arm,

TAAAGAAAC

SEQ ID NO: 241, RdRp (432/932), right arm,

TCCTTAAAG

SEQ ID NO: 242, RdRp (433/932), right arm,

TCCTTCCTT

SEQ ID NO: 243, RdRp (511/932), right arm,

TACCCCATT

SEQ ID NO: 244, RdRp (513/932), right arm,

AGCCTTACC

SEQ ID NO: 245, RdRp (522/932), right arm,

CATAACTCA

SEQ ID NO: 246, RdRp (524/932), right arm,

TGATCCTCA

SEQ ID NO: 247, RdRp (545/932), right arm,

TAAGATTCA

SEQ ID NO: 248, RdRp (549/932), right arm,

AATGGCATA

SEQ ID NO: 249, RdRp (553/932), right arm,

ATTCTTTGC

SEQ ID NO: 250, RdRp (553/932), right arm,

CTATTCTTT

SEQ ID NO: 251, RdRp (557/932), right arm,

ACGGTGCGA

SEQ ID NO: 252, RdRp (563/932), right arm,

ACAGATAGA

SEQ ID NO: 253, RdRp (583/932), right arm,

AGTGGCGGC

SEQ ID NO: 254, RdRp (584/932), right arm,

CTAGTGGCG

SEQ ID NO: 255, RdRp (801/932), right arm,

CAGTCCAAC

SEQ ID NO: 256, RdRp (875/932), right arm,

GATTAGGAT

SEQ ID NO: 257, RdRp (876/932), right arm,

CCTGATTAG

SEQ ID NO: 258, RdRp (919/932), right arm,

CAGGTTCCC

SEQ ID NO: 259, RdRp (922/932), right arm,

CATAAAACT

SEQ ID NO: 260, Helicase (202/601), right arm,

TTTTCAAAG

SEQ ID NO: 261, Helicase (261/601), right arm,

CATCTGAGA

SEQ ID NO: 262, Helicase (264/601), right arm,

AGAAAACTC

SEQ ID NO: 263, Helicase (288/601), right arm,

TACCAGTAC

SEQ ID NO: 264, S (918/1274), right arm,

CATAGAGAA

SEQ ID NO: 265, (8-17Dz5, left arm)

GCTG

SEQ ID NO: 266, (8-17Dz5, right arm)

GCTGC

SEQ ID NO: 267, (8-17Dz6, left arm)

TGCTG

SEQ ID NO: 268, (8-17Dz6, right arm)

GCTGCT

SEQ ID NO: 269, (8-17Dz7, left arm)

CTGCTG

SEQ ID NO: 270, (8-17Dz7, right arm)

GCTGCTG

SEQ ID NO: 271, (8-17Dz8, left arm)

GCTGCTG

SEQ ID NO: 272, (8-17Dz8, right arm)

GCTGCTGC

SEQ ID NO: 273, (8-17Dz9, left arm)

TGCTGCTG

SEQ ID NO: 274, (8-17Dz9, right arm)

GCTGCTGCT

SEQ ID NO: 275, (8-17Dz10, left arm)

CTGCTGCTG

SEQ ID NO: 276, (8-17Dz10, right arm)

GCTGCTGCTG

SEQ ID NO: 277, (8-17Dz11, left arm)

GCTGCTGCTG

SEQ ID NO: 278, (8-17Dz11, right arm)

GCTGCTGCTGC

SEQ ID NO: 279, (8-17Dz12, left arm)

TGCTGCTGCTG

SEQ ID NO: 280, (8-17Dz12, right arm)

GCTGCTGCTGCT

SEQ ID NO: 281, (8-17Dz13, left arm)

CTGCTGCTGCTG

SEQ ID NO: 282, (8-17Dz13, right arm)

GCTGCTGCTGCTG

SEQ ID NO: 283, (LNA8-17Dz9, left arm)

+T+G+C+T+GCTG

SEQ ID NO: 284, (LNA8-17Dz9, right arm)

GCTG+C+T+G+C+T

SEQ ID NO: 285, (PNA8-17Dz9, left arm)

*T*G*C*T*G*C*T*G

SEQ ID NO: 286, (PNA8-17Dz9, right arm)

*G*C*T*G*C*T*G*C*T

SEQ ID NO: 287, (8-17 catalytic core)

TCCGAGCCGGACGA

SEQ ID NO: 288,

CAGCAGCAGCAGCAGCAGCAGCAGCAG

SEQ ID NO: 289,

AGCAGCAGCAGCAGCAGCA

SEQ ID NO: 290,

AGTTTTTCTCATGGTGTATTTATTCTTTTAAGTTTTGTTTTTTAAATATA
CTTCACTTTTGAATGTTTCAGACAGCAGCAAAAGCAGCAACAGCAGCAGC
AGCAGCAGCAGCAGGGGGACATATCAGGACAGAGTTCACATCCATGTGAA

SEQ ID NO: 291, (10-23 catalytic core)

GGCTAGCTACAACGA

1. An engineered nucleic acid, comprising:
a first binding arm,
a catalytic core domain, and
a second binding arm,
wherein the catalytic core domain is in between the first binding arm and the second binding arm, and wherein the first binding arm and the second binding arm are complementary to a CAG repeat sequence.

2. The engineered nucleic acid of claim 1, wherein the catalytic core domain comprises a sequence at least 80% identical to SEQ ID NO: 287.

3. (canceled)

4. The engineered nucleic acid of claim 1, wherein the first binding arm and the second binding arm comprise at least 3 nucleotides.

5. The engineered nucleic acid of claim 4, wherein the first and the second binding arms comprise 6, 7, 8, 9, 10, 11, 12, or 13 nucleotides.

6. (canceled)

7. (canceled)

8. The engineered nucleic acid of claim 1, wherein the first binding arm and the second binding arm comprise a sequence at least 80% identical to SEQ ID NOs: 265-286.

9. The engineered nucleic acid of claim 1, wherein at least one nucleotide of the engineered nucleic acid is a chemically modified ribose.

10. The engineered nucleic acid of claim 1, wherein at least one nucleotide at the 5′-terminus and at least one nucleotide at the 3′-termius of the engineered nucleic acid are chemically modified.

11. The engineered nucleic acid of claim 9,wherein the chemically modified ribose is a locked nucleic acid (LNA) or a peptide nucleic acid (PNA).

12. The engineered nucleic acid of claim 1, wherein the engineered nucleic acid comprises a sequence at least 80% identical to SEQ ID NOs: 121-132.

13. A method of treating a polyglutamine disease in subject in need thereof comprising administering to the subject in need thereof a therapeutically effective amount of the engineered nucleic acid of claim 1.

14. The method of claim 13, wherein the engineered nucleic acid decreases a level of a polyglutamine disease-related polynucleotide.

15. The method of claim 14, wherein the polyglutamine disease-related polynucleotide comprises an increased level of CAG repeat sequence as compared to a reference control.

16. The method of claim 14 , wherein the polyglutamine disease-related polynucleotide is selected from a huntingtin (HTT) polynucleotide, an ATXN1 polynucleotide, an ATXN2 polynucleotide, an ATXN7 polynucleotide, a TATA binding protein-coding polynucleotide, an androgen receptor (AR) polynucleotide, and an atrophin 1 (ATN1) polynucleotide.

17. The method of claim 14, wherein the polyglutamine disease is selected from Huntington’s disease (HD), spinocerebellar ataxias (SCA) type 1, SCA type 2, SCA type 3, SCA type 6, SCA type 7, SCA type 17, Dentatorubral-pallidoluysian atrophy (DRPLA), and spinobulbar muscular atrophy (SBMA).

18. An engineered nucleic acid, comprising:
a first binding arm,
a catalytic core domain, and
a second binding arm,
wherein the catalytic core domain is in between the first binding arm and the second binding arm, and wherein the first binding arm and the second binding arm are complementary to a SARS-CoV-2 polynucleotide.

19-24. (canceled)

25. The engineered nucleic acid of claim claim 18, wherein the first binding arm and the second binding arm comprise a sequence at least 80% identical to SEQ ID NOs: 203-264.

26-28. (canceled)

29. The engineered nucleic acid of claim 18, wherein the engineered nucleic acid comprises a sequence at least 80% identical to SEQ ID NOs: 172-202.

30. A method of treating a SARS-CoV-2 infection in a subject in need thereof, comprising administering to the subject in need thereof a therapeutically effective amount of the engineered nucleic acid of claim 18.

31. The method of claim 30, wherein the engineered nucleic acid decreases a level of a SARS-CoV-2 polynucleotide.
